(19) United States
(12) Patent Application Publication
(10) Pub. No.: US 2026/0357344 A1
(43) Pub. Date: Nov. 12, 2026

(54) METHOD FOR CHARACTERISING THE DEGRADATION OF AN ORGAN

(71) Applicants: CGENETIX, Paris (FR); INSTITUT NATIONAL DE LA SANTÉ ET DE LA RECHERCHE MÉDICALE, Paris (FR); ASSISTANCE PUBLIQUE HÔPITAUX DE PARIS, PARIS Cedex 12 (FR); SORBONNE UNIVERSITÉ, PARIS (FR); UNIVERSITÉ PARIS CITÉ, PARIS (FR); CENTRE NATIONAL DE LA RECHERCHE SCIENTIFIQUE, PARIS (FR)

(72) Inventors: Geoffroy POULET, PARIS (FR); Pierre GALICHON, PARIS (FR); Dany ANGLICHEAU, PARIS (FR); Valérie TALY, BOURG-LA-REINE (FR); Hélène PÉRÉ, PARIS (FR); David VEYER, PARIS (FR); Pierre LAURENT-PUIG, MEUDON (FR); Guillaume BEINSE, PARIS (FR)

(73) Assignees: CGENETIX, Paris (FR); INSTITUT NATIONAL DE LA SANTÉ ET DE LA RECHERCHE MÉDICALE, PARIS (FR); ASSISTANCE PUBLIQUE HÔPITAUX DE PARIS, PARIS Cedex 12 (FR); SORBONNE UNIVERSITÉ, PARIS (FR); UNIVERSITÉ PARIS CITÉ, PARIS (FR); CENTRE NATIONAL DE LA RECHERCHE SCIENTIFIQUE, PARIS (FR)

(21) Appl. No.: 19/112,730

(22) PCT Filed: Sep. 18, 2023
(86) PCT No.: PCT/EP2023/075645

§ 371 Date: Mar. 18, 2025

(30) Foreign Application Priority Data
Sep. 19, 2022 (FR) FR2209432

(51) Int. Cl. C12Q 1/6883; C12Q 1/6809; C12Q 1/6827; C12Q 1/6844

(52) U.S. Cl. CPC C12Q 1/6883; C12Q 1/6809; C12Q 1/6827; C12Q 1/6844; C12Q 2600/154

(57) ABSTRACT

A kit and an in vitro method for detecting and characterizing the degradation of a particular organ or tissue, based on the analysis of the presence of at least one specific methylated cell-free DNA in a biological sample. More particularly, the kit and the method for detecting and characterizing the degradation of an organ selected from the brain, the lung and the kidney, and/or the degradation of a tissue present in this organ. Also, a method for the in vitro diagnosis of a disease or of a condition involving the lysis of an organ or a tissue.

### FIELD

The object of the present invention is a kit and an in vitro method for detecting and characterising the degradation of a particular organ or tissue, based on the analysis of the presence of at least one specific methylated cell-free DNA in a biological sample. More particularly, the object of the invention is a kit and a method for detecting, and characterising the degradation 1) of the brain during an episode of stroke, 2) of the lung during an episode of acute respiratory distress syndrome and 3) of the kidney during an episode of acute kidney injury and/or an episode of rejection and/or dysfunction of the kidney graft. Another object of the invention is a method for the in vitro diagnosis of a disease or of a condition involving the lysis of an organ or a tissue.

The present invention is therefore in the field of molecular biology applied to medical diagnostics.

### BACKGROUND

During the treatment of a patient affected by a disease in the acute phase, medical imaging analysis, such as a CT scan, an X-ray or an MRI, is performed in order to establish the overall level of suffering and of lesional damage of the patient. However, this type of examination does not make it possible to distinguish the organic lysis per se from inflammatory disease. However, the prognosis and therapeutic intervention performed by medical staff are very different according to the nature of the organ or tissue involvement. In addition, in the case of an acute phase, treatment decisions must be made within an extremely short time frame.

Some acute and relatively widespread diseases have the common feature of involving a massive tissue lysis.

This is in particular the case of stroke, wherein the brain tissue is degraded, acute respiratory distress syndrome, wherein the lung tissue is degraded, acute kidney injury (AKI) of a native kidney and a transplanted kidney when transplant rejection or kidney transplant dysfunction occurs, wherein the kidney tissue is degraded.

Moreover, in the case of kidney transplantation, it is essential to monitor the possible occurrence of graft rejection/dysfunction. Currently, the postoperative follow-up of transplant patients involves painful and burdensome monitoring for the patient.

Due to the incidence of all of these diseases among the population, there is a need for tools making it possible to reliably, quickly, simply and inexpensively detect possible tissue damage in the context of these various acute diseases.

Circulating cell-free DNA, also designated as “free DNA”, is generally known as a tumour marker, but also an inflammation marker and tissue stress marker. The immune cells contribute mostly to increasing the total amount of circulating DNA.

It has been shown that circulating DNA may possibly be used as a potential biomarker for autoimmune diseases of the systemic lupus emphysematous type or rheumatoid arthritis (Duvvuri et al., “Cell-free DNA as a biomarker in autoimmune rheumatic diseases”, Front. Immunol. 10, 2019).

The use, in a healthy subject, of the characterisation of the methylation profile of circulating DNA to identify the tissue origin of said circulating DNA has been described (Moss et al “Comprehensive human cell-type methylation atlas reveals origins of circulating cell-free DNA in health and disease”, Nat. Commun., 9, 1-12, 2018).

The increase in the proportion of circulating DNA from hepatocytes in patients with inflammatory liver disease has been described, the immune cells contributing mostly to the amount of total amount of circulating DNA (Liu et al (“Comprehensive DNA methylation analysis of tissue of origin of plasma cell-free DNA by methylated CpG tandem amplification and sequencing”, Clin. Epigenetics, 11, 93 2019).

WO 2021/216985 “Methods for detecting tissue damage, graft versus host disease and infections using cell-free DNA profiling” discloses the detection of the degradation of tissue by establishing the profile of circulating DNA in the context of the transplantation of allogeneic hematopoietic cells.

Document CN 113999901 “Myocardial specific methylation marker” describes the identification and the use of a specific cardiac methylated cell DNA marker for the diagnosis of acute myocardial infarction, from a plasma sample.

WO2019012544 (A1) “DUAL-PROBE DIGITAL DROPLET PCR STRATEGY FOR SPECIFIC DETECTION OF TISSUE-SPECIFIC CIRCULATING DNA MOLECULES” describes a droplet PCR method for analysing the methylation state of methylation sites of a double strand DNA molecule that comprises at least two single strand methylation sites of the double strand DNA molecule from a sample of a biological fluid such as plasma or urine

WO2019012543 (A1) “DNA TARGETS AS TISSUE-SPECIFIC METHYLATION MARKERS” describes a general method for determining the state of degradation of tissue during a pathological process from the study of the methylation sites of a circulating DNA molecule from a biological fluid such as plasma or urine.

### SUMMARY

The inventors developed a kit and a method for the specific and targeted detection of circulating methylated cell-free DNA present in a biological sample of an individual and specific to the brain, the lung, or the kidney. A method according to the invention makes it possible to specifically detect the existence of a massive lysis of one of these organs or of a particular tissue that composes it at the level of the vascular or epithelial fraction thereof. In addition, this process makes it possible to distinguish the lysis of an organ from an inflammatory phenomenon of immune origin. The distinction of epithelial or vascular damage as well as the identification of an organic lysis rather than an inflammatory phenomenon of purely immune origin is major for the diagnosis of the patient and cannot be discriminated by medical imaging.

For two of these organs, namely the lung and the brain, the inventors indeed selected, from various specifically hypermethylated or hypomethylated regions of circulating DNA, at least one region making it possible to develop a sensitive and specific test of the degradation of said organ.

For one of these organs, namely the kidney, the inventors further selected, from various fractions specifically hypermethylated or hypomethylated of circulating DNA, at least three fractions differentially methylated in the renal epithelium, the renal endothelium and the total kidney, these regions may be designated as respectively “total kidney”, the “renal vascular fraction” and the “renal epithelial fraction” making it possible to develop a sensitive and specific test of the degradation of said organ.

Thus, the inventors designed, for each of these regions, a pair of selective amplification primers, amplification conditions and a probe specifically detecting the amplification product generated using said pair of primers.

The inventors also identified, around each of the nucleotide sequences selectively amplified by a pair of primers as described above, a genetic region of interest including the amplified nucleotide sequence, said genetic region being larger than that of the marker. The present invention describes said “genetic regions of interest”.

For each of the markers, a detection kit and a detection method are thus provided.

Another object of the invention is a kit and a method for detecting at least one specific marker of the lysis of an organ. An additional object of the invention is a kit and a method for simultaneously detecting at least two markers for specifically detecting the lysis of an organ, thus making it possible to further improve the specificity and sensitivity of the test. A test according to the invention also has a strong diagnostic performance.

The object of the invention is therefore a kit and a method for detecting the lysis of an organ selected from the brain, the lung and the kidney, from a biological sample of a patient. A kit and a method for detecting the brain lysis can be used, in particular, for the in vitro diagnosis of stroke. This kit and this method are based on the detection of at least one marker selected from “APC2” and “NBR1”. The “NBR1” marker is a marker also designated as “KRT33B”.

Another object of the invention is a kit and a method for detecting lung lysis, which can be used in particular for the in vitro diagnosis of acute respiratory distress or of a possible lung graft rejection. This kit and this method are based on the detection of the “SYNE1” marker. The “SYNE1” marker is a marker also designated as “LINC00336”.

Another object of the invention is a kit and a method for detecting kidney lysis, which can be used in particular for the in vitro diagnosis of acute kidney injury and, in the case of a transplanted patient, the rejection of the transplant. This kit and this method are based on the detection of at least one marker selected from: PAX2, PDE4D, CTDP1, ALDH1A1, ARID3A, GATA2, LOC124903692, NRN1, SEPT5-GP1BB, TNS2-AS1 RHBDF2, LINC01599, FLJ12825, ACSL5.

According to a particular embodiment, another object of the invention is a kit and a method for detecting kidney lysis, based on the combination of the multiplexing detection of at least:
one “total kidney” marker: selected from CTDP1
one “Vascular” type marker: selected from the markers ALDH1A1, ARID3A, GATA2, LOC124903692, NRN1, SEPT5-GP1BB, TNS2-AS1 RHBDF2 and LINC01599 and
one “epithelial” type marker: selected from the markers FLJ12825, ACSL5, PAX2 and PDE4D.

Increasing the number of markers detected simultaneously by multiplexing analysis guarantees better clinical performance of the test in terms of diagnosis, screening, therapeutic optimisation and patient follow-up.

Solid biopsy, or kidney puncture, is the reference method for diagnosing transplant rejection. It makes it possible to study certain anatomical structures of the graft at the renal cortex, located peripherally to the kidney. Depending on the Banff score, graft lesions can be segmented by “vascular” or “epithelial” typology. The study of these lesions combined with the observation of immune infiltrates makes it possible to make the typological diagnosis of kidney transplant rejection as follows: “no rejection”, “T-cell mediated rejection (TCMR)”, “Antibody-mediated rejection (ABMR)” and “Mixed rejection combining both types of rejection”. Depending on the type of rejection, the treatment options are different.

Diagnostic tests for transplant rejection are marketed, this concerns Allosure (CareDX) and Prospera™ (Natera). The new methodologies for detecting transplant rejection articulate the proposal thereof only on the prediction of the rejection or of the dysfunction of the kidney graft, without being able to characterise and diagnose the nature of the latter. Therefore, the use of kidney puncture remains current.

A kit and a method according to the invention therefore have the great advantage of offering a minimally invasive test, because it is performed from an easily collected, reliable, fast and inexpensive biological sample. Due to the minimally invasive nature thereof, a method according to the invention can easily, if necessary, be repeated at various stages of the disease, which makes it possible to reliably monitor the progression of said disease and ensures medical treatment adapted to each of these stages.

A kit and a method according to the invention have a major challenge for specialists and anatomical pathologists, because the typology of organ lesions, in particular the typology of the kidney graft lesions, determines the final diagnosis and therapeutic treatment of the patient.

The sensitivity and specificity of the test for the presence of each of the markers were verified. A kit and a method according to the invention further have the advantage of being reproducible, fast and inexpensive.

Another object of the invention is the use of methylated cell-free DNA as a marker of the degradation of the brain, of the kidney or of the lung, or of the degradation of a tissue of the brain, of the kidney or of the lung.

Another object of the invention is the primers and probes that can be used in a kit and a method according to the invention. In particular, the object of the invention is:
a sense primer comprising, or consisting of a nucleotide sequence selected from: SEQ ID No. 1, SEQ ID No. 4, SEQ ID No. 7, SEQ ID No. 10, SEQ ID No. 13, SEQ ID No. 19, SEQ ID No. 22, SEQ ID No. 25, SEQ ID No. 28, SEQ ID No. 31, SEQ ID No. 34, SEQ ID No. 37, SEQ ID No. 40, SEQ ID No. 43, SEQ ID No. 46, SEQ ID No. 49, SEQ ID No. 52, or a nucleotide sequence having at least 80% identity with said sequences;
an antisense primer comprising, or consisting of a nucleotide sequence selected from: SEQ ID No. 2, SEQ ID No. 5, SEQ ID No. 8, SEQ ID No. 11, SEQ ID No. 14, SEQ ID No. 20, SEQ ID No. 23, SEQ ID No. 26, SEQ ID No. 29, SEQ ID No. 32, SEQ ID No. 35, SEQ ID No. 38, SEQ ID No. 41, SEQ ID No. 44, SEQ ID No. 47, SEQ ID No. 50 or SEQ ID No. 53 or a nucleotide sequence having at least 80% identity with said sequences;
and a probe, optionally marked, comprising, or consisting of a nucleotide sequence selected from: SEQ ID No. 3, SEQ ID No. 6, SEQ ID No. 9, SEQ ID No. 12, SEQ ID No. 15, SEQ ID No. 21, SEQ ID No. 24, SEQ ID No. 27, SEQ ID No. 30, SEQ ID No. 33, SEQ ID No. 36, SEQ ID No. 39, SEQ ID No. 42, SEQ ID No. 45, SEQ ID No. 48, SEQ ID No. 51 and SEQ ID No. 54, or a nucleotide sequence having at least 80% identity with said sequences.

Finally, the object of the invention is an in vitro method for detecting a disease or a condition selected from: stroke, acute respiratory distress syndrome, kidney injury and transplant rejection, in particular kidney or lung transplant rejection.

### BRIEF DESCRIPTION OF THE DRAWINGS

Other advantages and features will become apparent upon examining the detailed description of a non-limiting embodiment, and of the appended drawings, wherein:

FIG. 1 shows the proportion of neurons marked during the use of the anti-NeuN marker (left), of the APC2 marker (centre), and of the NBR1 marker (right).

FIG. 2 shows the normalised data of the mean of the NBR1 and APC2 markers, on a logarithmic scale in the case of the TAI (left), and of the stroke (right).

FIG. 3 shows the amount of renal markers in plasma (in ng/ml) in the case of patients at high risk of transplant rejection (left histograms) or at low risk of transplant rejection (right histograms), for the PDE4D (white) and PAX2 (black) markers.

FIG. 4 shows the fraction of circulating DNA of renal origin, out of the total circulating DNA in the plasma (in %) in the case of patients at high risk of transplant rejection (left histograms) or at low risk of transplant rejection (right histograms), for the PDE4D (white) and PAX2 (black) markers.

FIG. 5 shows the amount of renal markers in ng/ml of urine in the case of patients with transplant rejection (left histograms) or without transplant rejection (right histograms), for the PDE4D (white) and PAX2 (black) markers.

FIG. 6 shows the correlation between the TTF-1 histological marking (x-axis) and the use of the SYNE1 marker (y-axis) according to the invention.

FIG. 7 shows the discrimination between the subgroups of patients with moderate ARDS (left), severe ARDS (centre), or critical ARDS (right) based on the results obtained with the SYNE1 marker (ordinates).

FIG. 8 shows: (A) the detection of the markers SEPT5/GATA2, PDE4D, CTDP1, PAX2 and TNS2-AS1/ACSL5 in 100% methylated DNA; (B) the detection of the markers SEPT5/GATA2, PDE4D, CTDP1, PAX2 and TNS2-AS1/ACSL5 in non-methylated DNA, that is to say the negative control, the results showing no detection in non-methylated DNA.

FIG. 9 shows the relative quantification of the markers SEPT5, GATA2, TNS2-AS1, PDE4D, ACSL5, PAX2 and CTDP1, with normalisation by the albumin ubiquitous marker (internal control corresponding to the number of total genomes present in the sample) in genomic DNA (gDNA) samples extracted from healthy tissues: kidney, liver, white blood cells, plasma circulating DNA and urine circulating DNA.

FIG. 10 shows the correlation (R2=0.5) between the normalised relative quantifications (via an albumin internal marker) of the PAX2 and ACSL5 markers in n=22 renal gDNA.

FIG. 11 shows the correlation (R2=0.8087) between the normalised relative quantifications (via an albumin internal marker) of the PAX2 and ACSL5 markers in n=15 urine cf-DNA.

FIGS. 12A and 12B, respectively show the comparison of 2 quantitative methods of the renal epithelial fraction on n=22 healthy kidney samples. CD10 is a histological marker of the renal epithelium. PAX2 and ACSL5 are molecular markers specifically targeting the renal epithelium.

FIG. 13 shows the significant difference in quantification of the CTDP1 marker in urine between patients having a transplant rejection and those not having kidney transplant rejection (left histogram) and a logistic regression model compiling quantitative data of the biomarkers CTDP1, PAX2, PDE4D, ACSL5 and SEPT5 to predict the presence or the absence of transplant rejection (right graph)

FIG. 14 shows the significant difference in quantification of the CTDP1 biomarker in plasmas between patients having a transplant rejection (with tissue lesions on the graft) and those not having kidney transplant rejection (without tissue lesions on the graft) (left histogram) and a logistic regression model compiling quantitative data of the biomarkers CTDP1, GATA2, TNS2-AS1, PAX2 and PDE4D to predict the presence or the absence of transplant rejection (right graph).

FIG. 15 shows the significant difference in quantification of the GATA2 biomarker in plasmas between patients having vascular graft lesions and those not having vascular graft lesions (left graph) and a logistic regression model compiling quantitative data from the biomarkers CTDP1, GATA2, and TNS2-AS1 to predict the presence or the absence of vascular lesions on the graft (right graph).

### DETAILED DESCRIPTION

According to a first aspect, the object of the invention is a kit for detecting in a biological sample at least one methylated cell-free DNA target nucleotide sequence, said nucleotide sequence being specific to an organ selected from: the brain, the lung and the kidney or to a tissue present in the brain, the lung or the kidney, said kit comprising at least one pair of primers consisting of a sense primer and an antisense primer, each of the primers comprising, or consisting of, a nucleotide sequence capable of hybridising with a nucleotide sequence of said cell-free DNA to selectively amplify a specific methylated cell-free DNA sequence of said organ or tissue.

According to one embodiment of this first aspect, the object of the invention is a kit for detecting in a biological sample at least one methylated cell-free DNA target nucleotide sequence, said kit comprising at least:
one pair of primers consisting of a sense primer and an antisense primer, each of the primers comprising, or consisting of, a nucleotide sequence capable of hybridising with at least one nucleotide sequence of said cell-free DNA to selectively amplify a specific methylated cell-free DNA sequence of said organ or tissue, and
one probe comprising, or consisting of, a nucleotide sequence capable of hybridising with the target nucleotide sequence of amplified DNA.

“Biological sample” means an element from the body, human or animal, in particular for example: blood, urine, saliva, plasma or an organ fragment.

Said probe, so-called “detection probe”, is preferably associated with a marker the detection of which is well known to a person skilled in the art. When more than one pair of probes is used (multiplex test), the combination of markers is selected in order to distinguish the various amplified nucleotide sequences.

According to a first particular aspect, the object of the present invention is a kit for detecting in a biological sample a methylated cell-free DNA target nucleotide sequence, said nucleotide sequence being specific to a cell type of an organ selected from: the brain, the lung and the kidney, said kit comprising:
i) for detecting a specific cell-free DNA target sequence of a type of cells present in the brain, at least: one sense primer comprising a nucleotide sequence SEQ ID No. 1, or a nucleotide sequence having at least 80% identity with SEQ ID No. 1 and one antisense primer, comprising a nucleotide sequence SEQ ID No. 2, or a nucleotide sequence having at least 80% identity with SEQ ID No. 2, and/or
   one sense primer comprising a nucleotide sequence SEQ ID No. 4, or a nucleotide sequence having at least 80% identity with SEQ ID No. 4 and one antisense primer, comprising a nucleotide sequence SEQ ID No. 5, or a nucleotide sequence having at least 80% identity with SEQ ID No. 5.
ii) for detecting a specific cell-free DNA target sequence of a type of cells present in the lung, at least: one sense primer, comprising a nucleotide sequence SEQ ID No. 7, or a nucleotide sequence having at least 80% identity with SEQ ID No. 7 and one antisense primer, comprising a nucleotide sequence SEQ ID No. 8, or a nucleotide sequence having at least 80% identity with SEQ ID No. 8, and
iii) for detecting a specific cell-free DNA target sequence of a type of cells present in the kidney, at least:
   one sense primer comprising a sequence SEQ ID No. 10, or a sequence having at least 80% identity with SEQ ID No. 10 and one antisense primer, comprising a sequence SEQ ID No. 11, or a sequence having at least 80% identity with SEQ ID No. 11, and/or
   one sense primer comprising a sequence SEQ ID No. 13, or a sequence having at least 80% identity with SEQ ID No. 13 and one antisense primer, comprising a sequence SEQ ID No. 14, or a sequence having at least 80% identity with SEQ ID No. 14, and/or
   one sense primer comprising a sequence SEQ ID No. 19, or a sequence having at least 80% identity with SEQ ID No. 19 and one antisense primer, comprising a sequence SEQ ID No. 20, or a sequence having at least 80% identity with SEQ ID No. 20, and/or
   one sense primer comprising a sequence SEQ ID No. 22, or a sequence having at least 80% identity with SEQ ID No. 22 and one antisense primer, comprising a sequence SEQ ID No. 23, or a sequence having at least 80% identity with SEQ ID No. 23, and/or
   one sense primer comprising a sequence SEQ ID No. 25, or a sequence having at least 80% identity with SEQ ID No. 25 and one antisense primer, comprising a sequence SEQ ID No. 26, or a sequence having at least 80% identity with SEQ ID No. 26, and/or
   one sense primer comprising a sequence SEQ ID No. 28, or a sequence having at least 80% identity with SEQ ID No. 28 and one antisense primer, comprising a sequence SEQ ID No. 29, or a sequence having at least 80% identity with SEQ ID No. 29, and/or
   one sense primer comprising a sequence SEQ ID No. 31, or a sequence having at least 80% identity with SEQ ID No. 31 and one antisense primer, comprising a sequence SEQ ID No. 32, or a sequence having at least 80% identity with SEQ ID No. 32, and/or
   one sense primer comprising a sequence SEQ ID No. 34, or a sequence having at least 80% identity with SEQ ID No. 34 and one antisense primer, comprising a sequence SEQ ID No. 35, or a sequence having at least 80% identity with SEQ ID No. 35, and/or
   one sense primer comprising a sequence SEQ ID No. 37, or a sequence having at least 80% identity with SEQ ID No. 37 and one antisense primer, comprising a sequence SEQ ID No. 38, or a sequence having at least 80% identity with SEQ ID No. 38, and/or
   one sense primer comprising a sequence SEQ ID No. 40, or a sequence having at least 80% identity with SEQ ID No. 40 and one antisense primer, comprising a sequence SEQ ID No. 41, or a sequence having at least 80% identity with SEQ ID No. 41, and/or
   one sense primer comprising a sequence SEQ ID No. 43, or a sequence having at least 80% identity with SEQ ID No. 43 and one antisense primer, comprising a sequence SEQ ID No. 44, or a sequence having at least 80% identity with SEQ ID No. 44, and/or
   one sense primer comprising a sequence SEQ ID No. 46, or a sequence having at least 80% identity with SEQ ID No. 46 and one antisense primer, comprising a sequence SEQ ID No. 47, or a sequence having at least 80% identity with SEQ ID No. 47, and/or
   one sense primer comprising a sequence SEQ ID No. 49, or a sequence having at least 80% identity with SEQ ID No. 49 and one antisense primer, comprising a sequence SEQ ID No. 50, or a sequence having at least 80% identity with SEQ ID No. 50, and/or
   one sense primer comprising a sequence SEQ ID No. 52, or a sequence having at least 80% identity with SEQ ID No. 52 and one antisense primer, comprising a sequence SEQ ID No. 53, or a sequence having at least 80% identity with SEQ ID No. 53.

According to a more particular aspect, a kit according to the invention comprises at least:
one, two, three, four, five, six, seven, eight, nine, ten, eleven, twelve, thirteen, fourteen, fifteen, sixteen, seventeen, eighteen, nineteen or twenty pairs of primers, each consisting of a sense primer according to the invention and an anti-sense primer according to the invention.

According to a more particular aspect, a kit according to the invention comprises a “sense” primer comprising or consisting of, a nucleotide sequence selected from SEQ ID No. 1, SEQ ID No. 4, SEQ ID No. 7, SEQ ID No. 10, SEQ ID No. 13, SEQ ID No. 19, SEQ ID No. 22, SEQ ID No. 25, SEQ ID No. 28, SEQ ID No. 31, SEQ ID No. 34, SEQ ID No. 37, SEQ ID No. 40, SEQ ID No. 43, SEQ ID No. 46, SEQ ID No. 49, SEQ ID No. 52, or a nucleotide sequence comprising at least 80% identity with one of said sequences SEQ ID No. 1, SEQ ID No. 4, SEQ ID No. 7, SEQ ID No. 10, SEQ ID No. 13, SEQ ID No. 19, SEQ ID No. 22, SEQ ID No. 25, SEQ ID No. 28, SEQ ID No. 31, SEQ ID No. 34, SEQ ID No. 37, SEQ ID No. 40, SEQ ID No. 43, SEQ ID No. 46, SEQ ID No. 49 or SEQ ID No. 52.

According to another more particular aspect, a kit according to the invention comprises an “antisense” primer comprising or consisting of, a nucleotide sequence selected from SEQ ID No. 2, SEQ ID No. 5, SEQ ID No. 8, SEQ ID No. 11, SEQ ID No. 14, SEQ ID No. 20, SEQ ID No. 23, SEQ ID No. 26, SEQ ID No. 29, SEQ ID No. 32, SEQ ID No. 35, SEQ ID No. 38, SEQ ID No. 41, SEQ ID No. 44, SEQ ID No. 47, SEQ ID No. 50 or SEQ ID No. 53, or a nucleotide sequence comprising at least 80% identity with one of said nucleotide sequences SEQ ID No. 2, SEQ ID No. 5, SEQ ID No. 8, SEQ ID No. 11, SEQ ID No. 14, SEQ ID No. 20, SEQ ID No. 23, SEQ ID No. 26, SEQ ID No. 29, SEQ ID No. 32, SEQ ID No. 35, SEQ ID No. 38, SEQ ID No. 41, SEQ ID No. 44, SEQ ID No. 47, SEQ ID No. 50 or SEQ ID No. 53.

According to another more particular aspect, a kit according to the invention comprises a probe comprising or consisting of, a nucleotide sequence selected from SEQ ID No. 3, SEQ ID No. 6, SEQ ID No. 9, SEQ ID No. 12, SEQ ID No. 15, SEQ ID No. 21, SEQ ID No. 24, SEQ ID No. 27, SEQ ID No. 30, SEQ ID No. 33, SEQ ID No. 36, SEQ ID No. 39, SEQ ID No. 42, SEQ ID No. 45, SEQ ID No. 48, SEQ ID No. 51 and SEQ ID No. 54, or a nucleotide sequence comprising at least 80% identity with one of said nucleotide sequences selected from SEQ ID No. 3, SEQ ID No. 6, SEQ ID No. 9, SEQ ID No. 12, SEQ ID No. 15, SEQ ID No. 21, SEQ ID No. 24, SEQ ID No. 27, SEQ ID No. 30, SEQ ID No. 33, SEQ ID No. 36, SEQ ID No. 39, SEQ ID No. 42, SEQ ID No. 45, SEQ ID No. 48, SEQ ID No. 51 and SEQ ID No. 54.

“At least 80% identity” means that said sequences have at least 80% identity after optimal global alignment, that is to say by global alignment between two sequences giving the highest percentage of identity between them. The optimal global alignment of two sequences can in particular be performed according to the Needleman-Wunsch algorithm, well known to the person skilled in the art (Needleman & Wunsch, “A general method applicable to the search for similarities in the amino acid sequences of two proteins”, J. Mol. Biol., 48 (3): 443-53). A nucleotide sequence according to the invention comprises, or consists of a nucleotide sequence having at least 80%, advantageously at least 81%, 82%, 83%, 84%, 85%, 86%, 87%, 88%, 89%, 90%, 91%, 92%, 93%, 94%, 95%, 96%, 97%, 98% or 99% identity with the reference nucleotide sequence after optimal overall alignment. Advantageously, a nucleotide sequence according to the invention comprises, or consists of a nucleotide sequence having at least 99.1%, 99.2%, 99.3%, 99.4%, 99.5%, 99.6%, 99.7%, 99.8% or 99.9% identity with the reference nucleotide sequence after optimal overall alignment.

According to this aspect, a nucleotide sequence according to the invention comprising, or consisting of, a sequence having at least 80% identity with the reference nucleotide sequence is functional, that is to say that it is capable of hybridising on the target nucleotide sequence with sufficient stability to make the amplification or the detection of said target nucleotide sequence possible.

The nucleotide sequences of a first group of primers are designated as indicated in Table 1 below:

TABLE 1

| | Sense primer | | Antisense primer | |
|---|---|---|---|---|
| | SEQ | | SEQ | |
| | ID | | ID | |
| Marker | No. | Sequence | No. | Sequence |
| NBR1 | 1 | TCGGGTTTTCGGGTAACGT | 2 | CTTCGCCACGCAACAAC |
| APC2 | 4 | CGTAGGGTCGGAAGGAGG | 5 | ATCCGAACGAACGACGAA |
| SYNE1 | 7 | GTTAGATCGTAGGTTGCGC | 8 | ACGCGAACTCGAACACTT |
| PDE4D | 10 | GGTTTTAGCGGATGGCGA | 11 | GCTCCTCTACGAAACGAACA |
| PAX2 | 13 | GGCGAAATTCGGTGTATATAATTT | 14 | GAACGACAAAAACTCTACGAAA |
| Albumin | 16 | GGGATGGAAAGAATTTTATGTT | 17 | AAACAAACTAACCCCAAATTCT |

The nucleotide sequences of a first group of probes are designated as indicated in Table 2 below:

TABLE 2

| | Detection probe | |
|---|---|---|
| Marker | SEQ ID No. | Sequence |
| NBR1 | 3 | TGAAGAGTTGGTAGCGT |
| APC2 | 6 | CGTTTAAGGTTGTATTAGTTG |
| SYNE1 | 9 | CGCGGCGTAGGTATA |
| PDE4D | 12 | TGGCGAGCGCGTT |
| PAX2 | 15 | TCGTTTCGTTTCGTTCG |
| Albumin | 18 | AGGGTTTTTTATTATTATT |

According to another more particular aspect, the object of the invention is a kit for detecting in a biological sample a methylated cell-free DNA target nucleotide sequence, said nucleotide sequence being specific to a type of cells present in the brain, said kit comprising at least:
one sense primer comprising, or consisting of, a nucleotide sequence SEQ ID No. 1, or a nucleotide sequence having at least 80% identity with SEQ ID No. 1 and one antisense primer, comprising, or consisting of, a nucleotide sequence SEQ ID No. 2, or a nucleotide sequence having at least 80% identity with SEQ ID No. 2, and/or
one sense primer comprising, or consisting of, a nucleotide sequence SEQ ID No. 4, or a nucleotide sequence having at least 80% identity with SEQ ID No. 4 and one antisense primer, comprising, or consisting of, a nucleotide sequence SEQ ID No. 5, or a nucleotide sequence having at least 80% identity with SEQ ID No. 5.

According to another more particular aspect, the object of the invention is a kit for detecting in a biological sample a methylated cell-free DNA target nucleotide sequence, said nucleotide sequence being specific to a type of cells present in the lung, said kit comprising at least one sense primer comprising, or consisting of, a nucleotide sequence SEQ ID No. 7, or a nucleotide sequence having at least 80% identity with SEQ ID No. 7 and one antisense primer, comprising, or consisting of, a nucleotide sequence SEQ ID No. 8, or a nucleotide sequence having at least 80% identity with SEQ ID No. 8.

The nucleotide sequences of the specific primers of a type of cell present in the kidney are designated as indicated in Table 3 below:

TABLE 3

| | Sense primer | | Antisense primer | |
|---|---|---|---|---|
| | SEQ | | SEQ | |
| Marker | ID No. | Sequence | ID NO. | Sequence |
| FDE4D | 10 | GGTTTTAGCGGATGGCGA | 11 | GCTCCTCTACGAAACGAACA |
| PAX2 | 13 | GGCGAAATTCGGTGTATATAATTT | 14 | GAACGACAAAAACTCTACGAAA |
| FLJ1232S | 19 | TCGGGTTAGGTTTTGTTAAGGTA | 20 | TCCTCCGAAACGAAACGAAA |
| ALDH1A1 | 22 | TTTAAATCGGTGAGTAGGATGG | 23 | ATTCCAAAAACCGAATCAAAAATATCA |
| | | T | | |
| ARID3A | 25 | GCGGTTGTGTTTGAGATAGAG | 26 | CAAAACTAACGACCGAAAACC |
| ACSL5 | 28 | GGATATTTTGGGTCGGTTTTTTGT | 2 | CTCGCTCGTAATTCCTCCAAATCCCTCG |
| | | TGGGTCGGTTTTTTGTTTGTATG | | CTCGTAATTCCTC |
| CTDP1 | 31 | GAAACGGTGAGATGTTCGGAGG | 32 | CTTTACTATTACGAAACGTTCGTAATACA |
| | | GTTTAAATGAAACGGTGAGATGT | | CGAAACGTTCCTAATACAACAA |
| GATA2 | 34 | TTGGTTTTCGAATTTTGTTTGTTT | 35 | CTAATAATAAACTCCAAATCTACGCA |
| | | ATA | | |
| LOC124903892 | 37 | TGGGGGAGGGAAGGTCG | 38 | CTATAAAAAACAAATAAACTAAAAAACAC |
| | | | | GACAAATAAACTAAAAAACACGATCGA |
| NRN1 | 40 | AGTGTGGGTGGGCGCG | 41 | TCGACAACTTTAACGACCTAACTTATCTC |
| | | CGCGGGGGGAGTTGGT | | TCTCGACAACTTTAACGA |
| SEPT5- | 41 | CGTTTGTTGTTTTATTTGGTCGA | 44 | ACAAAACGAACCGAAAACGCCGCGTACA |
| GP185 | | TGGTCGAGGACGAGTTGC | | ACAACCCAAAA |
| TNS2-AS1 | 45 | AGTTATTTTCGGGTGTTTCGTTCG | 47 | CGAATCCAAACTAAAACTCCTAAAACGCT |
| | | GGTGTTTCGTTTTTGCG | | CCATAAACGAATCCAAAC |
| RHBDF2 | 48 | TCGTATTTGTTTTTATTTTGGTTGT | 50 | CTTTTCCCGAATAAACACCG |
| LINC01599 | 52 | ATTTATATTAGGTAGTGAGAAGAT | 53 | AATATAAATACCAAAACAAAAACACACAA |
| | | GTGT | | |

The nucleotide sequences of the probes specific to a type of cell present in the kidney are designated as indicated in Table 4 below:

TABLE 4

| | Detection probe | |
|---|---|---|
| | SEQ | |
| | ID | |
| Marker | NO | Sequence |
| PDE4D | 12 | TGGCGAGCGCGTT |
| PAX2 | 15 | TCGTTTCGTTTCGTTCG |
| FLJ12825 | 21 | TGGGGTTTTTGTGCGCGT |
| ALDH1A1 | 24 | AGTTTGGCGTGTTTGAG |
| ARID3A | 27 | TGTTAAGAGAGTTTCGAGT |
| ACSL5 | 30 | TGTATGGACGTTTTGAAGTTACGTTTTGAAGT |
| | | TATTTTGTTT |
| CTOP1 | 33 | AAGCGTCGGTTGTTTGTCGGAGGAAGCGTCGG |
| GATA2 | 38 | ACGAATTTTAGTACGGGGT |
| LOC124303692 | 39 | TGCGGTTTTGGTATCGAACGTGCGGTTTTGGT |
| NRN1 | 42 | AGTTGGTTTTACGTTTAGAAGTTACGTTTAGA |
| | | AGGAAAGTTAG |
| SEPT5- | 45 | ACGAGTTGCGCGTCGT CGTGTTTGCGTTTTC |
| GP188 | | G |
| TN52- | 48 | TGCGTTTATGCGTAGGTTTGCGTAGGTTTTTT |
| AS1 | | TTTTTTAG |
| RHBDF2 | 51 | TAAATTTCGGTTTCGGTTCG |
| LINC01599 | 54 | TGGTTTTGAGTTGATGGTAG |

From the tissues present in the kidney, the following can be cited: the renal epithelium tissue, and the renal vascular tissue

The markers dedicated to monitoring the kidney damage are characterised as indicated in Table 5 below:

TABLE 5

| Organ fraction | | | Chromosome position | |
|---|---|---|---|---|
| (Total, epithelial, | | | Chromosome | |
| vascular) | Type | Markers | no. | Position bases: start-end |
| Epithelial | Hypermethylated | FLJ12825 | 12 | 54,106,357-54,106,526 |
| Vascular | Hypermethylated | ALDH1A1 | 9 | 72,952,933-72,953,059 |
| Vascular | Hypermethylated | ARID3A | 19 | 940,667-941,160 |
| Epithelial | Hypermethylated | ACSL5 | 10 | 112,376,275-112,376,398 |
| Total kidney | Hypermethylated | CTDP1 | 18 | 79,791,700-79,791,820 |
| Vascular | Hypermethylated | GATA2 | 3 | 128,491,794-128,492,245 |
| Vascular | Hypermethylated | LOC124903692 | 16 | 54,937,300-54,937,500 |
| Vascular | Hypermethylated | NRN1 | 6 | 5,998,924-5,999,075 |
| Epithelial | Hypermethylated | PAX2 | 10 | 100,828,800-100,829,020 |
| Epithelial | Hypermethylated | PDE4D | 5 | 59,039,300-59,039,420 |
| Vascular | Hypermethylated | SEPT5-GP1BB | 22 | 19,724,235-19,724,340 |
| Vascular | Hypermethylated | TNS2-AS1 | 12 | 53,054,207-53,054,329 |
| Vascular | Hypermethylated | RHBDF2 | 17 | 76,500,822-76,500,937 |
| Vascular | Hypomethylated | LINC01599 | 14 | 50,056,666-50,056,758 |

According to another more particular aspect, the object of the invention is a kit that contains the reagents necessary (primers and probes) for the amplification of at least one PCR amplicon of less than <85 bp (preferably <70 bp) in a genomic region comprised from the nucleotide sequences SEQ ID No. 55, SEQ ID No. 56, SEQ ID No. 57, SEQ ID No. 58, SEQ ID No. 59, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70 and SEQ ID No. 71 according to the methylation status of said genomic region.

The markers used in a kit and a method according to the invention are therefore likely to be classified as:
“vascular type” markers: selected from the markers ALDH1A1, ARID3A, GATA2, LOC124903692, NRN1, SEPT5-GP1BB, TNS2-AS1 RHBDF2 and LINC01599,
“epithelial type” markers: selected from the markers FLJ12825, ACSL5, PAX2 and PDE4D,
“total kidney type” markers: selected from CTDP1.

According to another more particular aspect, the object of the invention is a kit for detecting in a biological sample a methylated cell-free DNA target nucleotide sequence, said nucleotide sequence being specific to a type of cells present in the kidney, said kit comprising at least:
one sense primer comprising, or consisting of, a nucleotide sequence likely to detect a “total kidney type” marker and one antisense primer, comprising, or consisting of, a nucleotide sequence likely to detect a “total kidney type” marker, and/or
one sense primer comprising, or consisting of, a nucleotide sequence likely to detect a “vascular type” marker and one antisense primer, comprising, or consisting of, a nucleotide sequence likely to detect a “vascular type” marker, and/or
one sense primer comprising, or consisting of, a nucleotide sequence likely to detect an “epithelial type” marker and one antisense primer, comprising, or consisting of, a nucleotide sequence likely to detect an “epithelial type” marker.

Preferably, according to this more particular aspect, the object of the invention is a kit for detecting in a biological sample a methylated cell-free DNA target nucleotide sequence, said nucleotide sequence being specific to a type of cells present in the kidney, said kit comprising at least:
one sense primer comprising, or consisting of, a nucleotide sequence likely to detect a “total kidney type” marker and one antisense primer, comprising, or consisting of, a nucleotide sequence likely to detect a “total kidney type” marker, said marker being CTDP1 and
one sense primer comprising, or consisting of, a nucleotide sequence likely to detect a “vascular type” marker and one antisense primer, comprising, or consisting of, a nucleotide sequence likely to detect a “vascular type” marker, said marker being selected from GATA2, TNS2-AS1 and
one sense primer comprising, or consisting of, a nucleotide sequence likely to detect an “epithelial type” marker and one antisense primer, comprising, or consisting of, a nucleotide sequence likely to detect an “epithelial type” marker, said marker being selected from: PAX2, ACSL5 or PDE4D.

The markers used in a kit and a method according to the invention will now be described in more detail. The marker names correspond to the name of the gene whereon these markers are located.

The genetic region “PDE4D” according to the invention designates a sequence coding for a protein of the phosphodiesterase family. The DNA sequence thereof is located on chromosome 5 at position 58,969,038-60,522,128, more specifically at locus 5q11.2-q12.1. It contains 42 exons. The DNA sequence thereof is referenced as NC_000005.10. It is transcribed in the sequence NM_006203.5. The inventors identified the DNA region included in chromosome 5 position 59,039,300-59,039,420 (SEQ ID No. 55), as being specifically hyper-methylated in the epithelial fraction. They propose to use this gene in a non-invasive, sensitive and reliable method, in particular to diagnose or identify a kidney transplant rejection in the kidney transplant patient, or in a kit useful for such purposes.

The genetic region “PAX2” according to the invention designates a coding sequence for a protein of the box-paired family 2. The DNA sequence thereof is located on chromosome 10 at position 100,735,396-100,829,944, more specifically at locus 10q24.31. It contains 14 exons. The DNA sequence thereof is referenced as NC_000010.11. It is transcribed in the sequence NM_000278.5. The inventors identified the DNA region comprised in chromosome 10 position 100,828,800-100,829,020 (SEQ ID No. 56), as being specifically hyper-methylated in the epithelial fraction. In particular, they propose to use this gene in a non-invasive, sensitive and reliable method to diagnose or identify kidney transplant rejection in the kidney transplant patient, or in a kit useful for such purposes.

The genetic region “FLJ12825” according to the invention designates a non-coding complete genomic molecule for proteins. It makes it possible to transcribe non-coding long RNA and is present on the uncharacterised locus LOC440101 of the FLJ12825 gene, The DNA sequence thereof is located on chromosome 12 at position 54,058,254-54,122,234, more specifically at locus 12q13.13 in an intronic sequence. The DNA sequence thereof is referenced as NC_000012.12. It is transcribed into 1 RNA variant named NR_026655.1. The present inventors identified the DNA region included in chromosome 12: Position 54,106,357-54,106,526 (SEQ ID No. 57), as being specifically hyper-methylated in the renal epithelial fraction. In particular, they propose to use this gene in a non-invasive, sensitive and reliable method to diagnose or identify kidney transplant rejection in the kidney transplant patient, or in a kit useful for such purposes.

The genetic region “ALDH1A1” according to the invention designates an encoding complete genomic molecule for a protein of the aldehyde dehydrogenase family type 1 (member A1). The DNA sequence thereof is located on chromosome 9 at position 72,900,671-72,953,063, more specifically at locus 9q21.13. It contains 13 exons. The DNA sequence thereof is referenced as NC_005224.3. It is transcribed in 1 RNA variant named NM_005224.3. The present inventors identified the DNA region included in chromosome 9: Position 72,952,933-72,953,059 (SEQ ID No. 58), as being specifically hyper-methylated in the renal vascular fraction. They therefore propose to use this gene in a non-invasive, sensitive and reliable method, in particular to diagnose or identify kidney transplant rejection in the kidney transplant patient, or in a kit useful for such purposes.

The genetic region “ARID3A” according to the invention designates a coding complete genomic molecule for a protein of the ARID (AT-rich interaction domain) DNA-binding protein family. The DNA sequence thereof is located on chromosome 19 at position 925,732-975,939, more specifically at locus 19p13.3. It contains 12 exons. The DNA sequence thereof is referenced as NC_000019.10. It is transcribed in 1 RNA variant named NM_000689.5. The present inventors identified the DNA region included in chromosome 19: Position 940,667-941,160 (SEQ ID No. 59), as being specifically hyper-methylated in the renal vascular fraction. They therefore propose to use this gene in a non-invasive, sensitive and reliable method, in particular to diagnose or identify kidney transplant rejection in the kidney transplant patient, or in a kit useful for such purposes.

The genetic region “ACSL5” according to the invention designates a coding sequence for a protein of the ligase family. The DNA sequence thereof is located on chromosome 10 at position 112,374,116-112,428,376, more specifically at locus 10q25.2. It contains 23 exons. The DNA sequence thereof is referenced as NC_000010.11. It is transcribed in the sequence NM_016234.4. The present inventors identified the DNA region included in chromosome 10 position 112,376,275-112,376,398 (SEQ ID No. 60), as being specifically hyper-methylated in the epithelial fraction. They therefore propose to use this gene in a non-invasive, sensitive and reliable method, in particular to diagnose or identify kidney transplant rejection in the kidney transplant patient, or in a kit useful for such purposes.

The genetic region “CTDP1” according to the invention designates an uncharacterised intronic sequence located between the CTDP1 and KCNG2 gene. The DNA sequence thereof is located on chromosome 18 between the positions of the genes CTDP1 (chr18: 79,756,625-79,787,722) and KCNG2 (chr18: 79,797,938-79,900,100). It contains 0 exons. The DNA sequence thereof is not referenced in the NCBI. There is no transcript identified.

The present inventors identified the DNA region included in chromosome 18 position 79,791,700-79,791,820 (SEQ ID No. 61), as being specifically hyper-methylated in the total fraction of the kidney. They therefore propose to use this gene in a non-invasive, sensitive and reliable method, in particular to diagnose or identify kidney transplant rejection in the kidney transplant patient, or in a kit useful for such purposes.

The genetic region “GATA2” according to the invention designates a sequence coding for a protein of the family of zinc finger DNA transcription factors. The DNA sequence thereof is located on chromosome 3 at position 128,479,422-128,493,201, more specifically at locus 3q21.3. It contains 8 exons. The DNA sequence thereof is referenced as NC_000003.12. It is transcribed in the sequence NM_032638.5. The present inventors identified the DNA region included in chromosome 3 position 128,491,794-128,492,245 (SEQ ID No. 62), as being specifically hyper-methylated in the vascular fraction. They therefore propose to use this gene in a non-invasive, sensitive and reliable method, in particular to diagnose or identify kidney transplant rejection in the kidney transplant patient, or in a kit useful for such purposes.

The genetic region “LOC124903692” according to the invention designates an uncharacterised and non-coding sequence for proteins. The DNA sequence thereof is located on chromosome 16 at position 54,936,014-54,938,671, more specifically at locus 16q12.2. It contains 3 exons. The DNA sequence thereof is referenced as NC_000016.10. It is transcribed in the sequence XR_007065074.1. The present inventors identified the DNA region included in chromosome 16 position 54,937,300-54,937,500 (SEQ ID No. 63), as being specifically hyper-methylated in the vascular fraction. They therefore propose to use this gene in a non-invasive, sensitive and reliable method, in particular to diagnose or identify kidney transplant rejection in the kidney transplant patient, or in a kit useful for such purposes.

The genetic region “NRN1” according to the invention designates a coding sequence for a protein of the neuritin family. The DNA sequence thereof is located on chromosome 6 at position 5,997,999-6,007,518, more specifically at locus 6p25.1. It contains 7 exons. The DNA sequence thereof is referenced as NC_000006.12. It is transcribed in the sequence NM_016588.3. The present inventors identified the DNA region included in chromosome 6 position 5,998,924-5,999,075 (SEQ ID No. 64), as being specifically hyper-methylated in the vascular fraction. They therefore propose to use this gene in a non-invasive, sensitive and reliable method, in particular to diagnose or identify kidney transplant rejection in the kidney transplant patient, or in a kit useful for such purposes.

The genetic region “SEPT5-GP1BB” according to the invention designates a non-coding sequence for proteins. The DNA sequence thereof is located on chromosome 22 at position 19,717,220-19,724,774, more specifically at locus 22q11.21. It contains 12 exons. The DNA sequence thereof is referenced as NC_000022.11. It is transcribed in the sequence NR_037611.1. The present inventors identified the DNA region included in chromosome 22 position 19,724,235-19,724,340 (SEQ ID No. 65), as being specifically hyper-methylated in the vascular fraction. They therefore propose to use this gene in a non-invasive, sensitive and reliable method, in particular to diagnose or identify kidney transplant rejection in the kidney transplant patient, or in a kit useful for such purposes.

The genetic region “TNS2-AS1” according to the invention designates a non-coding sequence for proteins. The DNA sequence thereof is located on chromosome 12 at position 53,043,189-53,054,438, more specifically at locus 12q13.13. It contains 4 exons. The DNA sequence thereof is referenced as NC_000012.12. It is transcribed in the sequence NR_033854.1. The inventors identified the DNA region included in chromosome 12 position 53,054,207-53,054,329 (SEQ ID No. 66), as being specifically hyper-methylated in the vascular fraction. They therefore propose to use this gene in a non-invasive, sensitive and reliable method, in particular to diagnose or identify kidney transplant rejection in the kidney transplant patient, or in a kit useful for such purposes.

The genetic region “RHBDF2” according to the invention designates a coding sequence for a protein making it possible to activate the protein transporter activity. The DNA sequence thereof is located on chromosome 17 at position 76,470,893-76,501,427, more specifically at locus 17q25.1. It contains 21 exons. The DNA sequence thereof is referenced as NC_000017.11. It is transcribed in the sequence NM_024599.5. The inventors identified the DNA region included in chromosome 17 position 76,500,822-76,500,937 (SEQ ID No. 67), as being specifically hyper-methylated in the vascular fraction. They therefore propose to use this gene in a non-invasive, sensitive and reliable method, in particular to diagnose or identify kidney transplant rejection in the kidney transplant patient, or in a kit useful for such purposes.

The genetic region “LINC01599” according to the invention designates a sequence making it possible to transcribe non-coding intergenic RNA for proteins. The DNA sequence thereof is located on chromosome 14 at position 50,007,313-50,105,043, more specifically at locus 14q21.3. It contains 9 exons. The DNA sequence thereof is referenced as NC_000014.9. It is transcribed in the sequence NR_131171.1. The inventors identified the DNA region included in chromosome 14 position 50,056,666-50,056,758 (SEQ ID No. 68), as being specifically hypo-methylated in the vascular fraction. They therefore propose to use this gene in a non-invasive, sensitive and reliable method, in particular to diagnose or identify kidney transplant rejection in the kidney transplant patient, or in a kit useful for such purposes.

The genetic region “APC2” according to the invention designates a complete genomic molecule coding sequence for a protein of the APC regulator family of the Wnt pathway. The DNA sequence thereof is located on chromosome 19 at position 1,446,230-1,473,244, more specifically at locus 19p13.3. It contains 17 exons. The DNA sequence thereof is referenced as NC_000019.10. It is transcribed in the sequence NM_005883.3. The inventors identified the DNA region included in chromosome 19 position 1,467,853-1,468,054 (SEQ ID No. 69), as being specifically hyper-methylated in the neurons. They therefore propose to use this gene in a non-invasive, sensitive and reliable method, in particular to diagnose a stroke, or in a kit useful for such purposes. The genetic region “NBR1” according to the invention designates a coding complete genomic molecule for a protein of the specific autophagy receptor family. The DNA sequence thereof is located on chromosome 17 at position 43,170,409-43,211,900, more specifically at locus 17q21.31. It contains 23 exons. The DNA sequence thereof is referenced as NC_000017.11. It is transcribed in the sequence NM_005899.5. The inventors identified the DNA region included in chromosome 17 position 43,211,655-43,211,856 (SEQ ID No. 70), as being specifically hyper-methylated in the neurons. They therefore propose to use this gene in a non-invasive, sensitive and reliable method, in particular to diagnose a stroke, or in a kit useful for such purposes.

The genetic region “SYNE1” according to the invention designates a coding complete genomic molecule for a protein of the nuclear envelope containing repeated sequences of spectrin. The DNA sequence thereof is located on chromosome 6 at position 152,121,687-152,637,362, more specifically at locus 6p25.2. It contains 153 exons. The DNA sequence thereof is referenced as NC_000006.12. It is transcribed in the sequence NM_033071.5. The inventors identified the DNA region included in chromosome 6 position 152,302,152-152,302,353 (SEQ ID No. 71), as being specifically hyper-methylated in pulmonary pneumocytes/alveoli. They therefore propose to use this gene in a non-invasive, sensitive and reliable method, in particular to diagnose or identify ARDS, or in a kit useful for such purposes.

A kit according to the invention optionally comprises one or more pairs of primers consisting of a sense primer and an anti-sense primer, intended to detect the presence of a control element in the biological sample. This control element may be any suitable element selected by a person skilled in the art. Said control element may in particular be albumin. More particularly, a kit according to the invention may comprise one sense primer comprising, or consisting of, a nucleotide sequence SEQ ID No. 16, or a nucleotide sequence having at least 80% identity with SEQ ID No. 16 and one antisense primer, comprising, or consisting of, a nucleotide sequence SEQ ID No. 17, or a nucleotide sequence having at least 80% identity with SEQ ID No. 17.

According to a second aspect, the object of the invention is a method for identifying a specific cell-free DNA target sequence of a type of cells present in a first organ selected from: the lung, the brain and the kidney, the method comprising at least the following steps of:
a) Determining, in at least one genomic DNA methylation database, the position and the degree of methylation of the CpG islands of specific sequences of said cell type of said organ,
b) Identifying, in the genomic DNA of said cell type of said healthy organ, specifically hypermethylated CpG islands,
c) Comparing the degree of methylation of the hypermethylated CpG islands of said cell type of said first organ with the degree of methylation of the same CpG islands of a second cell type, different from the first cell type,
d) Selecting the CpG islands only hypermethylated in the genomic DNA of said cell type of said first organ after comparison performed in step c),
e) Comparing the degree of methylation of the hypermethylated CpG islands of said cell type of said first organ with the degree of methylation of the same CpG islands of the genomic DNA of the other organs of said first organ,
f) Comparing the degree of methylation of the hypermethylated CpG islands of said cell type of said first organ with the degree of methylation of the same CpG islands of the genomic DNA of the healthy white blood cells,
g) Comparing the degree of methylation of the hypermethylated CpG islands of said cell type of said first organ with the degree of methylation of the same CpG islands of the cell-free DNA of all biological matrices combined from healthy subjects,
h) Selecting the CpG islands only hypermethylated in the genomic DNA of said cell type of said first organ after comparison performed in step e, f and g)
i) Identifying one or more target hypermethylated sequences of specific cell-free DNA of said cell type of the first organ from the comparison performed in step h).

Another object of the invention is a methylated cell-free DNA target nucleotide sequence identified by means of a method according to the invention, for the use thereof in detecting the lysis of an organ selected from the brain, the lung and the kidney, from a biological sample of a patient.

According to a third aspect, the object of the invention is a method for detecting, in a biological sample, a methylated cell-free DNA target nucleotide sequence, said nucleotide sequence being specific to an organ selected from: the lung, the brain and the kidney, or to a tissue present in the brain, the lung or the kidney, said method comprising at least the following steps of:
a) Bringing together, under conditions suitable for an amplification of the nucleic acids, the cell-free DNA previously extracted from said biological sample and a pair of primers consisting of a sense primer and an antisense primer, each of the primers comprising, or consisting of, a nucleotide sequence capable of hybridising with a nucleotide sequence of said target sequence of cell-free DNA,
b) Amplifying the reaction of said target sequence,
c) Detecting the presence of the amplified sequence during step b).

The cell-free DNA is extracted using a commercial kit, well known to the person skilled in the art and requires the presence of at least 0.15 ng/ml of target DNA in the urine or plasma sample.

The amplification reaction is performed by means of any technique or protocol well known to the person skilled in the art. From the non-limiting examples of these techniques, mention may be made of: PCR amplification, digital PCR amplification (dPCR), next generation sequencing.

More particularly, the object of the invention is a method for detecting in a biological sample a methylated cell-free DNA target nucleotide sequence, said nucleotide sequence being specific to a type of cells present in the brain.

Even more particularly, the object of the invention is a method for detecting in a biological sample a methylated cell-free DNA target nucleotide sequence, said nucleotide sequence being specific to a type of cells present in the brain, said method comprising at least the following steps of:
a) Bringing together, under conditions suitable for an amplification of the nucleic acids, the cell-free DNA previously extracted from said biological sample and a pair of primers consisting of one sense primer comprising or consisting of a nucleotide sequence SEQ ID No. 1 (or a nucleotide sequence having at least 80% identity with SEQ ID No. 1) and an antisense primer SEQ ID No. 2 (or a nucleotide sequence having at least 80% identity with SEQ ID No. 2), or one sense primer comprising or consisting of a nucleotide sequence SEQ ID No. 4 (or a nucleotide sequence having at least 80% identity with SEQ ID No. 4) and an antisense primer SEQ ID No. 5 (or a nucleotide sequence having at least 80% identity with SEQ ID No. 5), each of the primers comprising, or consisting of, a nucleotide sequence capable of hybridising with a nucleotide sequence of said target sequence of cell-free DNA, or bringing together, under conditions suitable for an amplification of the nucleic acids,
b) Amplifying the reaction of said target sequence,
c) Detecting the presence of the amplified sequence during step b), by means of a probe comprising, or consisting of SEQ ID No. 3 or SEQ ID No. 6, respectively according to the pairs of primers used.

Even more particularly, the object of the invention is a method for detecting in a biological sample a methylated cell-free DNA target nucleotide sequence, said nucleotide sequence being specific to a type of cells present in the lung, said method comprising at least the following steps of:
Bringing together, under conditions suitable for an amplification of the nucleic acids, the cell-free DNA previously extracted from said biological sample and a pair of primers consisting of
a sense primer comprising or consisting of a nucleotide sequence SEQ ID No. 7 (or a nucleotide sequence having at least 80% identity with SEQ ID No. 7) and an antisense primer SEQ ID No. 8 (or a nucleotide sequence having at least 80% identity with SEQ ID No. 8),
each of the primers comprising, or consisting of, a nucleotide sequence capable of hybridising with a nucleotide sequence of said target sequence of cell-free DNA, or bringing together, under conditions suitable for an amplification of the nucleic acids,
b) Amplifying the reaction of said target sequence,
c) Detecting the presence of the amplified sequence during step b), by means of a probe comprising, or consisting of SEQ ID No. 9.

Even more particularly, the object of the invention is a method for detecting in a biological sample a methylated cell-free DNA target nucleotide sequence, said nucleotide sequence being specific to a type of cells present in the kidney, said method comprising at least the following steps of:
a) Bringing together, under conditions suitable for an amplification of the nucleic acids, the cell-free DNA previously extracted from said biological sample and a pair of primers consisting of
a sense primer comprising or consisting of a nucleotide sequence SEQ ID No. 10 (or a nucleotide sequence having at least 80% identity with SEQ ID No. 10) and an antisense primer SEQ ID No. 11 (or a nucleotide sequence having at least 80% identity with SEQ ID No. 11), or
a sense primer comprising or consisting of a nucleotide sequence SEQ ID No. 13 (or a nucleotide sequence having at least 80% identity with SEQ ID No. 13) and an antisense primer SEQ ID No. 14 (or a nucleotide sequence having at least 80% identity with SEQ ID No. 14), each of the primers comprising, or consisting of, a nucleotide sequence capable of hybridising with a nucleotide sequence of said target sequence of cell-free DNA, or bringing together, under conditions suitable for an amplification of the nucleic acids,
b) Amplifying the reaction of said target sequence,
c) Detecting the presence of the amplified sequence during step b), by means of a probe comprising, or consisting of SEQ ID No. 12 or SEQ ID No. 15, respectively according to the pairs of primers used.

The diagnostic tests described are part of an analytical process composed of 2 preliminary steps: the extraction of circulating DNA and the epigenetic conversion (by bisulfite, enzymatic reaction). The diagnostic tests are compatible with different circulating DNA extraction kits such as the QIAamp Circulating Nucleic Acid Kit (50) Cat. No./ID: 55114 (Qiagen); EZ1&2 ccfDNA Kit (48) Cat. No./ID: 954854 (Qiagen); MagMAX™ cell-free DNA Isolation Kit (Reference: A29319, Thermofisher); Automated High-Throughput Extraction of Cell-Free DNA from Plasma, Serum, Urine and CSF (Ref.: A6030, Promega). Once extracted, the cell-free DNA are assayed by Qubit 4.0™ Fluorometry (Invitrogen™ db HS Qubit™ DNA Assay Kits (Reference: Q32851)) then are subjected to bisulfite conversion with the commercial Zymo Research EZ DNA Methylation-Lightning Kit (Reference D5031) or enzymatic equivalent (NEBNext® Enzymatic Methyl-seq Conversion Module (Reference E7125S/E7125L)). The samples are subsequently analysed by droplet digital PCR (System Naica (STILLA Technologies)) (also compatible with commercial equivalents such as QIAcuity Digital PCR System-QIAGEN and QX ONE Droplet Digital PCR (ddPCR) System-Bio-Rad).

The diagnostic tests described are compatible with all digital PCR platforms on the market (microdroplets and/or micro-compartments). Among them, we find commercial equivalents such as the QIAcuity Digital PCR System-QIAGEN and QX ONE Droplet Digital PCR (ddPCR) System-Bio-Rad).

The inventors determine the typical metrological parameters, in particular the repeatability, the reproducibility and the sensitivity of the test. To quantify the kidney degradation, at least 1 of the markers is needed. Detection with two markers strengthens the biological data.

Preferably, to quantify the kidney degradation, it is preferable to detect at least one “total kidney” marker, one “vascular type” marker and one “epithelial type” marker quantified in a multiplexing test. The higher the number of markers in each of these categories, the higher the clinical performance of the test. The detection of multiple markers per category from the “total kidney”, “vascular type” and “epithelial type” markers strengthens the biological data.

According to another aspect, another object of the invention is the use of a kit or a method according to the invention, for specifically detecting the degradation of the brain and/or for the in vitro diagnosis and/or the prognosis, and/or monitoring the progression of a stroke and/or the therapeutic optimisation of the patient suffering from a stroke.

According to another aspect, another object of the invention is the use of a kit or a method according to the invention, for specifically detecting the degradation of the lung and/or for the in vitro diagnosis/or the prognosis, and/or monitoring the progression of an acute respiratory distress syndrome and/or the therapeutic optimisation of the patient suffering from an acute respiratory distress syndrome.

According to another aspect, another object of the invention also relates to the use of a kit or of a method according to the invention, for specifically detecting the degradation of the kidney and/or for the in vitro diagnosis and/or the prognosis, and/or screening and/or monitoring the progression of a kidney transplant rejection/dysfunction and/or of an episode of acute kidney injury and/or the therapeutic optimisation of the patient suffering from a kidney transplant rejection/dysfunction and/or from an acute kidney injury.

According to another aspect, another object of the invention is a nucleotide sequence selected from:
a sense primer comprising, or consisting of a nucleotide sequence selected from: SEQ ID No. 1, SEQ ID No. 4, SEQ ID No. 7, SEQ ID No. 10, SEQ ID No. 13, SEQ ID No. 19, SEQ ID No. 22, SEQ ID No. 25, SEQ ID No. 28, SEQ ID No. 31, SEQ ID No. 34, SEQ ID No. 37, SEQ ID No. 40, SEQ ID No. 43, SEQ ID No. 46, SEQ ID No. 49, SEQ ID No. 52, or a nucleotide sequence having at least 80% identity with said sequences;
an antisense primer comprising a nucleotide sequence selected from: SEQ ID No. 2, SEQ ID No. 5, SEQ ID No. 8, SEQ ID No. 11, SEQ ID No. 14, SEQ ID No. 20, SEQ ID No. 23, SEQ ID No. 26, SEQ ID No. 29, SEQ ID No. 32, SEQ ID No. 35, SEQ ID No. 38, SEQ ID No. 41, SEQ ID No. 44, SEQ ID No. 47, SEQ ID No. 50 and SEQ ID No. 53, or a nucleotide sequence having at least 80% identity with said sequences; and
a probe comprising a nucleotide sequence selected from: SEQ ID No. 3, SEQ ID No. 6, SEQ ID No. 9, SEQ ID No. 12, SEQ ID No. 15, SEQ ID No. 21, SEQ ID No. 24, SEQ ID No. 27, SEQ ID No. 30, SEQ ID No. 33, SEQ ID No. 36, SEQ ID No. 39, SEQ ID No. 42, SEQ ID No. 45, SEQ ID No. 48, SEQ ID No. 51 and SEQ ID No. 54, or a nucleotide sequence having at least 80% identity with said sequences.

According to another aspect, the object of the invention is to diagnose, or monitor the degradation of an organ selected from the brain, the lung and the kidney, comprising the following steps of:
Extracting the methylated cell-free DNA from a biological sample of an individual
Converting the methylated cell-free DNA (bisulfite and/or enzymatic conversion)
Bringing together the DNA and a suitable pair of primers under conditions allowing hybridisation,
Amplifying said sequence,
Detecting the amplified sequence, by means of the suitable detection probe.

### EXAMPLES

### Example 1: Biomarker Identification

The CpG islands present at the promoters of certain genes are found at different methylation levels according to the nature of the tissue. From the use of methylation databases TCGA (The Cancer Genome Atlas) and DeepBlue (Epigenomic Data Server-DeepBlue), Gene Expression Omnibus (ID: GSE186458) the positions and degrees of methylation of a large number of CpG islands are listed for many different tissue types, including pulmonary (lung) pneumocyte subtypes, neurons (brain), and renal (kidney) cells (Moss et al. Nat Commun. December 2018; 9 (1): 5068; Liu X et al. Clin Epigenetics. December 2019; 11 (1): 93; Silva T C et al. Bioinformatics. 1 Jun. 2019; 35 (11): 1974-7; Zhou W et al. Nucleic Acids Res. 24 Oct. 2016; gkw967; Albrecht F et al. Nucleic Acids Res. 8 Jul. 2016; 44 (W1): W581-6). Through bioinformatic processing, the inventors identified various hypermethylated positions in the healthy brain, lung, and kidney tissues. This identification includes 5 steps and is described below in the example of the detection of lung degradation.

In the case of the detection of lung degradation, the inventors selected non-methylated positions in the DNA from white blood cells, PBMCs and immune cells, based on an analysis of data from whole blood.

The inventors subsequently detected hypermethylated and/or hypomethylated CpG islands in healthy tissue, in a database established from healthy lung tissue. This data was separated into a discovery dataset (“training”) and a validation dataset (“test”).

Differently methylated CpG islands of lung tissue compared to CpG islands of healthy tissue of other organs (kidney, brain, muscle, stomach, liver, heart, intestine) were subsequently identified. The most differentially methylated positions were selected, therefore 432 positions were retained.

From these 432 positions, a refinement was performed by manual selection. Positions are retained according to their mean methylation in pulmonary and non-pulmonary tissues. 4 candidate positions were selected.

The performance of these four positions to discriminate pulmonary tissue from non-pulmonary tissue were analysed, using a penalised logistic regression model according to the LASSO method. The statistical AUROC test obtained makes it possible to conclude that a plurality of CpG islands make it possible to identify a pulmonary tissue with strong predictive performance.

Table 6 below summarises the markers selected for each target organ.

TABLE 6

| Biomarker | Organ | Cell type | Chromome position |
|---|---|---|---|
| SYNE1 | Lung | Pneumocyte | Chr6: 152302252-152302253 (Hg38) |
| NBR1 | Brain | Neuron | Chr17: 43211755-43211756 (Hg38) |
| APC2 | Brain | Neuron | Chr19: 1467953-1467954 (Hg38) |
| PAX2 | Kidney | Kidney | Chr10: 100828934-100828935 |
| | | epithelium | (Hg38) |
| PDE4D | Kidney | Kidney | Chr5: 59039353-59039354 (Hg38) |
| | | epithelium | |

Various amplicons were generated in order to discriminate each of the positions identified. The technical features of the primers and probes are described in Table 4 below. Probe hybridisation temperatures were selected between 40° and 54 °C for the probes and 52 and 60 °C for the primers, the hybridisation temperature difference between primers and probes is from 5 to 10 °C.

TABLE 7

| | | | | | | Hybridization |
|---|---|---|---|---|---|---|
| | Oligonucleotide | | Target | SEQUENCE (5′ → 3′) | | Temperature |
| Name | Type | Concentration | Organ | 8-30 bases | % GC | (in °C) |
| PDD | Probe | 100 | Kidney | TGGCGAGCGCGTT | 69% | 44 |
| PDE4D | Sense Primer | 200 | Kidney | GGTTTTAGCGGATGGCGA | 56% | 56 |
| PDE4D | Antisense Primer; | 200 | Kidney | GCTCCTCTACGAAACGAACA | 50% | 60 |
| SYNE1 | Probe | 100 | Lung | CGCGGCGTAGGTATA | 60% | 48 |
| SYNE1 | Sense Primer | 200 | Lung | GTTAGATCGTAGGTTGCGC | 53% | 58 |
| SYNE1 | Antisense Primer; | 200 | Lung | ACGCGAACTCGAACACTT | 50% | 54 |
| NBR1 | Probe | 100 | Brain | TGA AGA GTT GGT AGC GT | 36% | 50 |
| NBR1 | Sense Primer | 200 | Brain | TCGGGTTTTCGGGTAACGT | 53% | 58 |
| NBR1 | Antisense Primer; | 200 | Brain | CTTCGCCACGCAACAAC | 59% | 54 |
| APC2 | Probe | 100 | Brain | CGTTTAAGGTTGTATTAGTTG | 33% | 56 |
| APC2 | Sense Primer | 200 | Brain | CGTAGGGTCGGAAGGAGG | 67% | 60 |
| APC2 | Antisense Primer; | 200 | Brain | ATCCGAACGAACGACGAA | 50% | 54 |
| PAX2 | Probe | 100 | Kidney | TCGTTTCGTTTCGTTCG | 47% | 50 |
| PAX2 | Sense Primes | 200 | Kidney | GGCGAAATTCGGTGTATATAATTT | 32% | 64 |
| PAX2 | Antisense primer; | 200 | Kidney | GAACGACAAAAACTCTACGAAA | 35% | 60 |

For each organ, a multiplexing test was constructed. An internal control of the amount of total circulating DNA, detecting the amount of coding DNA for albumin, is implemented in the test.

### Example 2: Validation of the Biomarkers for Neuronal Degradation in Acute Stroke Treatment

The validation of the biomarkers according to the invention comprises the following steps of:
the molecular analysis of the conversion of the DNA in droplet digital PCR tests, —validating the digital PCR test on 100% methylated synthetic DNA and commercial non-methylated genomic DNA,
determining the metrological parameters,
a negative control demonstrating the specificity of the test,
a positive control on genomic DNA samples from healthy tissue sections, and comparison with the reference histological marking

The molecular analysis of the conversion of the DNA in droplet digital PCR tests is common to the various markers. The cell-free DNA is first extracted by a commercial extraction process dedicated to the cell-free DNA via QIAamp Circulating Nucleic Acid Kit (Reference Cat. No./ID: 55114, Qiagen). The cell-free DNA is assayed by Qubit 4.0™ Fluorimetry (Invitrogen™ Kit db HS Qubit™ DNA Assay Kits (Reference: Q32851)) then is subjected to a bisulfite conversion with the Zymo Research EZ DNA Methylation-Lightning Kit (Reference D5031) or commercial equivalent. The samples are subsequently analysed by droplet digital PCR (System Naica (STILLA Technologies)).

The design of the primers and probes required for the PCR reaction for the APC2 and NBR1 targets are mentioned in Table 7. Taqman-Mgb type probes are used (Reference: PB-MGBEF-006, PB-MGBEC-006 and PB-MGBEH-006, Eurogentec). The implementation of the PCR amplification reaction is carried out in a method comprising 3 main steps presented below, this method is designated as “PCR amplification according to the invention” in the remainder of the text of the present application.

The preparation of the specific primer-probe cocktail is presented in Table 8 below.

TABLE 8

| | Initial Conc. in | Final Conc.in | Volume | Conc. in 25 μL |
|---|---|---|---|---|
| | Parent Tube | Assay Mix | 1 Reaction | of reaction mix |
| Reactive PCR | (μM) | (μM) | (μL) | (μM) |
| Albumin sense primer | 200 | 16 | 0.1 | 0.8 |
| Albumin antisense primer | 200 | 16 | 0.1 | 0.8 |
| Albumin probe - (VIC) | 100 | 12 | 0.15 | 0.6 |
| APC2 sense primer | 200 | 12 | 0.075 | 0.6 |
| APC2 antisense primer | 200 | 12 | 0.075 | 0.6 |
| APC2 sensor (Cy5) | 100 | 5 | 0.0625 | 0.25 |
| NBR1 sense primer | 200 | 12 | 0.075 | 0.6 |
| NBR1 antisense primer | 200 | 12 | 0.075 | 0.6 |
| NBR1 probe (FAM) | 100 | 8 | 0.1 | 0.4 |
| QSP H20 | | | 0.4375 | |
| Total | | | 1.25 | |

The preparation of the PCR reaction mix is presented in Table 9 below.

TABLE 9

| | | Vol 1 |
|---|---|---|
| | | Reaction |
| Reaction mixture | [Final] | well (μL) |
| Master mix (PerfeCTa ® Multiplex qPCR | 5X | 5 |
| ToughMix Cat. number: 733-2324 (VWR) | | |
| Fluorescein (100 μM) Cat. number: | 25X | 1 |
| 0681-100 g (VWR) | | |
| Primer-probe cocktail (Brain specific) - | 20X | 1.25 |
| Table X | | |
| Qsp H20 | | 12.75 |

The PCR reaction is presented in Table 10.

TABLE 10

| | Temperature | | Number |
|---|---|---|---|
| Parameter | (°C) | Time | of cycles |
| Partition of the sample | Saphire V1-Generation program | | 1 |
| Enzyme initialisation | 95 | 3′ | 1 |
| Denaturation of the DNA | 94 | 30″ | 45 |
| Hybridisation of primers/probes | 58.2 | 1′ | 45 |

| | | |
|---|---|---|
| Release of the pressure | Saphire V1-Release program | 1 |

The plates are read according to the following acquisition setting: 120 ms FAM channel (blue)—180 ms HEX/VIC channel (green)—38 ms Cy5 channel (red)) for reading with the STILLA Technologies Naica system.

Validation of the digital PCR test: The digital PCR test is subsequently validated on 100% methylated synthetic DNA and on commercial non-methylated genomic DNA. 3 synthetic DNA samples (100% methylated, Universal Methylated DNA Standard, Zymo Research Reference: D5011) and 3 commercial genomic DNA samples (non-methylated, Promega G1521) were analysed as follows: Step 1: Bisulfite conversion by the Zymo Research EZ DNA Methylation-Lightning Kit (Reference D5031) then Step 2: Analysis by Naica System digital PCR of the NBR1 and APC2 neurological markers.

The results on the 100% methylated DNA controls show that the epigenetic markers targeted on the NBR1 and APC2 genes are well detected and quantifiable by the test according to the invention. The results on the non-methylated DNA controls show that the developed NBR1 and APC2 markers are well detectable if and only if the target regions are hypermethylated. There is no marker detection in non-methylated DNA controls.

The specificity of the test was verified by a control in the presence of DNA from peripheral blood mononuclear cells (PBMC) of healthy subjects.

10 genomic DNA samples were extracted from white blood cell pellets of healthy subjects via the Qiagen QIAamp DNA Blood Mini Kit (50) Cat. No./ID: 51104. The samples were subsequently subjected to a bisulfite conversion step by the Zymo Research EZ DNA Methylation-Lightning Kit (Reference D5031) then an analysis by Naica System digital PCR of the NBR1 and APC2 neurological markers according to the invention described.

The negative test results for the epigenetic markers tested show that the test is specific because it has no biological noise in the genomic DNA from white blood cells. “Biological noise” means background noise from epigenetic markers on samples considered negative or non-methylated. In other words, the detection of the NBR1 and APC2 neurological markers generates a negligible signal on samples considered non-negative or non-methylated.

The specificity of the test was also verified in the presence of plasma DNA of healthy subjects. 10 plasma DNA samples were extracted from 1 mL of plasma of healthy subjects via the Qiagen QIAamp Circulating Nucleic Acid Kit (50) Cat. No./ID: 55114. The samples were subsequently subjected to a bisulfite conversion step by the Zymo Research EZ DNA Methylation-Lightning Kit (Reference D5031) then an analysis by Naica System digital PCR of the NBR1 and APC2 neurological markers according to the invention described. The negative test results for the epigenetic markers tested show that the latter does not show any biological noise in the cell-free/plasma DNA from the plasma of healthy subjects.

Finally, the specificity of the test was determined in the presence of genomic DNA of other tissues. N=27 healthy tissue samples were collected (Transfer of biological samples MTA No. CT 69HCL22_0234 from the anatomopathology department of the Hospices Civiles de Lyon Sud et Est). For each sample, three 8 μm-thick tissue shavings prepared in paraffin were transferred. The genomic DNA of the brain-derived tissue shavings were extracted via the Qiagen QIAamp DNA FFPE Advanced Kit (50) (Cat. No./ID: 56604). The DNA samples were subsequently subjected to a bisulfite conversion step by the Zymo Research EZ DNA Methylation-Lightning Kit (Reference D5031) then an analysis by Naica System digital PCR of the NBR1 and APC2 neurological markers according to the invention described. The results show very low biological noise in genomic DNA tissues other than brain-derived tissues. If the two APC2 and NBR1 biomarkers are taken into account, the biological noise is less than 2%. The test results for the tested epigenetic markers show that it has negligible or no biological noise at the genomic DNA from the various tissue sections (other than the brain).

A positive control is performed on genomic DNA samples from tissue sections of healthy brain tissues, by comparison with anti-NeuN histological marking.

12 healthy brain tissue samples were collected (Transfer of biological samples MTA no. VAL 2022/2022-234/01 from the anatomopathology department of hôpital Lariboisière du Pr Homa Adle Biassette). 1 sample=White slide marked with the anti-neuN antibody (neural antibody, Ref ABN91, Aldricht-Merck sigma) and three 8 μm-thick tissue shavings prepared in paraffin.

The neural cells marked with the anti-NeuN antibody were counted by the “HALO® image analysis” precision software. The genomic DNA of the brain-derived tissue shavings was extracted via the “Qiagen QIAamp DNA FFPE Advanced Kit” (50) (Cat. No./ID: 56604). The DNA samples were subsequently subjected to a bisulfite conversion step by the Zymo Research EZ DNA Methylation-Lightning Kit (Reference D5031) then an analysis by Naica System digital PCR of the NBR1 and APC2 neurological markers according to the invention described.

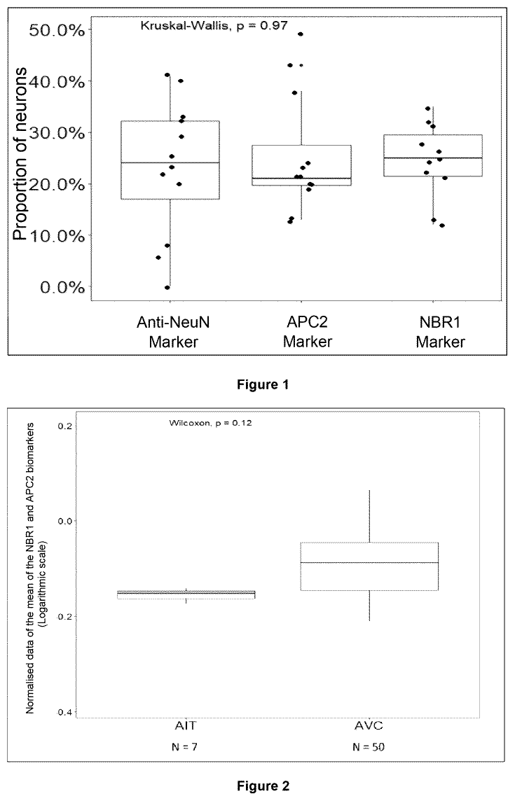

The APC2 and NBR1 neural markers are identified in 12 samples out of 12 samples tested. An identical proportion of neurons in healthy brain tissue sections is observed between the anti-NeuN histological marker and the APC2 and NBR1 epigenetic markers. A correlation between the APC2/NBR1 epigenetic markers and the anti-NeuN histological marker is noted (Pearson coefficient R=0.44 and 0.46 respectively for the APC2 and NBR1 epigenetic markers compared to the anti-NeuN marker). The results are shown in Table 11 below and in FIG. 1.

TABLE 11

| | | Anti- | | % | |
|---|---|---|---|---|---|
| | Hematox | NeuN | | Neurones - | % |
| | Positive | Positive | Total | Anti-NeuN | NBR1 |
| Sample | Cells | Cells | cells | (+) | (+) |
| LB1800307 | 224417 | 91369 | 315786 | 28.934% | 34.97% |
| LB1811732 | 283429 | 82933 | 366362 | 22.637% | 25.57% |
| LB1814745 | 140357 | 97715 | 238072 | 41.044% | 22.43% |
| LB2009543 | 73736 | 4692 | 78428 | 5.983% | 23.79% |
| LB2102358 | 126971 | 83593 | 210564 | 39.700% | 25.47% |
| LB2110295 | 100374 | 33345 | 133719 | 24.937% | 30.61% |
| LB2110560 | 233863 | 57881 | 291744 | 19.840% | 20.93% |
| LB2200857 | 170959 | 82397 | 253356 | 32.522% | 32.22% |
| LB2200948 | 273380 | 24149 | 297529 | 8.117% | 12.57% |
| LB2201563 | 177752 | 50297 | 228049 | 22.055% | 28.23% |
| LB2203330 | 164890 | 76015 | 240905 | 31.554% | 12.70% |

The correlation data between the quantitative assay of brain markers (neurons) and the Anti-NeuN histological marking (neural cell marker) show a correlation between the molecular and histological markings (Pearson coefficient R=0.46 and 0.44 respectively for NBR1 and APC2) confirming the neural specificity (brain cells) of the test.

In a clinical application, a test according to the invention makes it possible to stratify patients with stroke according to the severity index thereof. The results show an elevation in the quantitative mean of the APC2 and NBR1 markers in patients with acute ischaemic stroke (AIS) compared to patients with transient ischaemic stroke (TIA) (mean respectively of 11.32 copies/mL of plasma for TIA versus 75 copies/mL of plasma for AIA). The results are shown in FIG. 2.

### Example 3: Validation of a First Group of Biomarkers for Kidney Degradation in the Treatment of the Kidney Transplant Patient

The validation of PDE4D and PAX2 biomarkers comprises the molecular analysis of DNA conversion to droplet digital PCR tests, the design of Taqman-mgb primers and probes (Reference: PB-MGBEF-006, PB-MGBEC-006 and PB-MGBEH-006, Eurogentec) are identical to that described in Example 2 (see: Analysis methodology common part). The implementation of the amplification reaction by PCR comprises preparing the specific primer-probe cocktail is presented in the following Table 12.

TABLE 12

| | Initial Conc. in | Final Conc. in | Volume | Conc. in 25 μL |
|---|---|---|---|---|
| | Parent Tube | Assay Mix | 1 Reaction | of reaction mix |
| Reactive PCR | (μM) | (μM) | (μL) | (μM) |
| Albumin sense primer | 200 | 8 | 0.05 | 0.4 |
| Albumin antisense primer | 200 | 8 | 0.05 | 0.4 |
| Albumin probe - (VIC) | 100 | 12 | 0.15 | 0.6 |
| Pax2 sense primer | 200 | 12 | 0.075 | 0.6 |
| Pax2 antisense primer | 200 | 12 | 0.075 | 0.6 |
| Pax2 sensor (Cy5) | 100 | 8 | 0.1 | 0.4 |
| PDE4D sense primer | 200 | 12 | 0.075 | 0.6 |
| PDE4D antisense primer | 200 | 12 | 0.075 | 0.6 |
| PDE4D probe (FAM) | 100 | 8 | 0.1 | 0.4 |
| QSP H20 | | | 0.5 | |
| Total | | | 1.25 | |

The preparation of the PCR reaction mix is presented in Table 13 below.

TABLE 13

| | | Vol 1 |
|---|---|---|
| | | Reaction |
| Reaction mixture | [Final] | well (μL) |
| Master mix (PerfeCTa ® Multiplex qPCR | 5X | 5 |
| ToughMix Cat. number: 733-2324 (VWR) | | |
| Fluorescein (100 μM) Cat. number: | 25X | 1 |
| 0681-100 g (VWR) | | |
| Primer-probe cocktail (kidney specific) - | 20X | 1.25 |
| Table X | | |
| Qsp H20 | | 12.75 |

The PCR reaction is performed as indicated in Table 10 (Example 2). The plates are read as indicated in Example 2.

The digital PCR test is subsequently validated on 100% methylated synthetic DNA and on commercial non-methylated genomic DNA. 3 synthetic DNA samples (100% methylated, Universal Methylated DNA Standard, Zymo Research Reference: D5011) and 3 commercial genomic DNA samples (non-methylated, Promega G1521) were analysed as follows: Step 1: Bisulfite conversion by the Zymo Research EZ DNA Methylation-Lightning Kit (Reference D5031) then Step 2: Naica System digital PCR analysis of PDE4D and Pax2 renal markers. The results on synthetic DNA show that: 1/On the 100% methylated DNA controls: the epigenetic markers targeted on the PDE4D and Pax2 genes are well detected and quantifiable by our test. 2/On the non-methylated DNA controls: The developed PDE4D and Pax2 markers are well detectable if and only if the target regions are hypermethylated. There is no marker detection in non-methylated DNA controls.

The results show that the PDE4D and PAX2 targets are not detected in commercial non-methylated DNA.

The inventors determine typical metrological parameters, in particular the repeatability and the sensitivity of the test. The test is repeatable with a coefficient of variation of 4.5% and 0.6% respectively for the Pax2 and PDE4D markers. The test is sensitive to 0.01% and makes it possible to quantify up to a limit of 0.15 ng and 0.3 ng of DNA respectively for the PDE4D and Pax2 markers.

The specificity of the test was verified in the presence of DNA from PBMC (white blood cells) of healthy subjects. The markers are not detected during the analysis in PBMC DNA.

10 genomic DNA samples were extracted from white blood cell pellets of healthy subjects via the Qiagen QIAamp DNA Blood Mini Kit (50) Cat. No./ID: 51104. The samples were subsequently subjected to a bisulfite conversion step by the Zymo Research EZ DNA Methylation-Lightning Kit (Reference D5031) then an analysis by Naica System digital PCR of the PDE4D and Pax2 renal markers according to the invention described. The markers are not detected when analysing genomic DNA from white blood cell pellets of healthy subjects (=no biological background noise).

The specificity of the test was also verified in the presence of plasma DNA of healthy subjects. 10 plasma DNA samples were extracted from 1.5 mL of plasma of healthy subjects via the Qiagen QIAamp Circulating Nucleic Acid Kit (50) Cat. No./ID: 55114. The samples were subsequently subjected to a bisulfite conversion step by the Zymo Research EZ DNA Methylation-Lightning Kit (Reference D5031) then an analysis by Naica System digital PCR of the PDE4D and Pax2 renal markers according to the invention described. The markers are not detected during the analysis in the plasma DNA (cellular DNA) of healthy subjects.

Finally, the specificity of the test was determined in the presence of genomic DNA of other tissues. N=27 healthy tissue samples were collected (Transfer of biological samples MTA No. CT 69HCL22_0234 from the anatomopathology department of the Hospices Civiles de Lyon Sud et Est and of Hopital Lariboisière). For each sample, three 8 μm-thick tissue shavings prepared in paraffin were transferred. The genomic DNA from the tissue shavings were extracted via the Qiagen QIAamp DNA FFPE Advanced Kit (50) (Cat. No./ID: 56604). The samples were subsequently subjected to a bisulfite conversion step by the Zymo Research EZ DNA Methylation-Lightning Kit (Reference D5031) then an analysis by Naica System digital PCR of the PDE4D and Pax2 renal markers according to the invention described. The negative test results for the PDE4D and Pax2 epigenetic markers show that these were not detected in genomic DNA from tissue sections other than the kidney confirming the absence of biological noise.

A positive control is performed on genomic DNA samples from tissue sections of healthy kidney tissues. 12 samples of healthy kidney tissues were collected from the anatomopathology department of the Hospices Civils de Lyon Sud et Est. The genomic DNA from the tissue shavings were extracted via the Qiagen QIAamp DNA FFPE Advanced Kit (50) (Cat. No./ID: 56604). The samples were subsequently subjected to a bisulfite conversion step by the Zymo Research EZ DNA Methylation-Lightning Kit (Reference D5031) then an analysis by Naica System digital PCR of the PDE4D and Pax2 renal markers according to the invention described.

The PDE4D and Pax2 renal markers are identified in 12 samples out of 12 samples tested. Thus, this work confirms the kidney specificity of the PDE4D and Pax2 digital PCR test described.

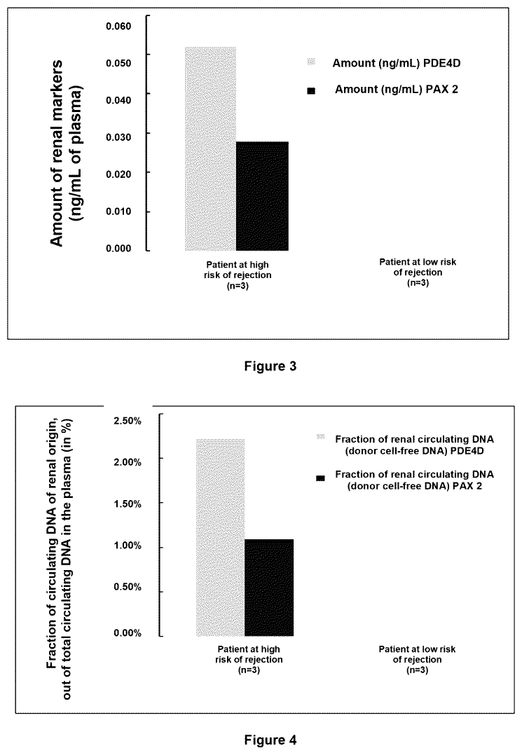

The amount of circulating DNA of renal origin is higher in the plasma of patients having a high risk of transplant rejection versus the plasma of patients having a low risk of transplant rejection during the first days post-kidney transplant (FIG. 3).

The fraction of circulating DNA of renal origin is higher in the plasma of patients having a high risk of transplant rejection versus the plasma of patients having a low risk of transplant rejection during the first days post-kidney transplant (FIG. 4).

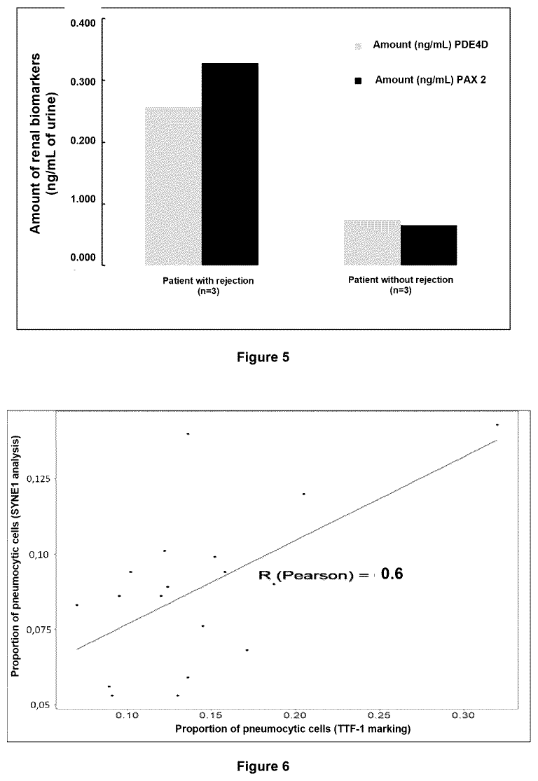

The quantitative assay of the PD4ED and Pax2 markers in urine by the described test is higher in patients with a transplant rejection confirmed by biopsy than in patients not having transplant rejection. This assay makes it possible to stratify the patients with a transplant rejection confirmed by biopsy and the patients not having transplant rejection (FIG. 5).

### Example 4: Validation of the SYNE1 Biomarker for Lung Degradation in the Treatment of Acute Respiratory Failure

The molecular analysis methodology of DNA conversion to droplet digital PCR tests, the design of the Taqman-mgb primers and probes (Reference: PB-MGBEF-006, PB-MGBEC-006 and PB-MGBEH-006, Eurogentec) are identical to that described in Example 2. The implementation of the PCR amplification reaction is carried out as follows. The preparation of the specific primer-probe cocktail is presented in Table 14 below.

TABLE 14

| | | | Volume for | C in 25 μL |
|---|---|---|---|---|
| | InitialC | FinalC | 1 reactions | emulsion |
| Reagents | (μM) | (μM) | (μL) | (μM) |
| Primer FORWARD ALB | 200 | 8 | 0.09 | 0.4 |
| Primer REVERSE ALB | 200 | 8 | 0.09 | 0.4 |
| Probe ALB-VIC | 100 | 12 | 0.055 | 0.6 |
| Primer FORWARD SYNE1 | 200 | 12 | 0.09 | 0.6 |
| Primer REVERSE SYNE1 | 200 | 12 | 0.09 | 0.6 |
| Probe SYNE1 - CY5 | 100 | 8 | 0.085 | 0.4 |
| H20 | | | 0.7 | |

The preparation of the PCR reaction mix is presented in Table 15 below.

TABLE 15

| | | Vol 1 |
|---|---|---|
| | | Reaction |
| Reaction mixture | [Final] | well (μL) |
| Master mix (PerfeCTa ® MultiPlex qPCR | 5X | 5 |
| ToughMix Cat. number: 733-2324 (VWR) | | |
| Fluorescein (100 μM) Cat. number: | 25X | 1 |
| 0681-100 g (VWR) | | |
| Primer-probe cocktail (lung specific) - | 20X | 1.25 |
| Table X | | |
| Qsp H20 | | 12.75 |

The PCR reaction is performed as indicated in Table 10 (Example 2). The plates are read as indicated in Example 2.

The digital PCR test is subsequently validated on 100% methylated synthetic DNA and on commercial non-methylated genomic DNA. 3 synthetic DNA samples (100% methylated, Universal Methylated DNA Standard, Zymo Research Reference: D5011) and 3 commercial genomic DNA samples (non-methylated, Promega G1521) were analysed as follows: Step 1: Bisulfite conversion by the Zymo Research EZ DNA Methylation-Lightning Kit (Reference D5031) then Step 2: Naica System digital PCR analysis of the SYNE1 pulmonary marker.

The results on the 100% methylated DNA controls show that the epigenetic marker targeted on the SYNE1 gene is well detected and quantifiable by a test according to the invention implementing this marker. The results on the non-methylated DNA controls show that the developed SYNE1 marker is well detectable if and only if the target regions are hypermethylated. There is no marker detection in the non-methylated DNA controls.

The specificity of the test was verified in the presence of genomic DNA from PBMC (white blood cells) of healthy subjects. 10 genomic DNA samples were extracted from white blood cell pellets of healthy subjects via the Qiagen QIAamp DNA Blood Mini Kit (50) Cat. No./ID: 51104. The samples were subsequently subjected to a bisulfite conversion step by the Zymo Research EZ DNA Methylation-Lightning Kit (Reference D5031) then an analysis by Naica System digital PCR of the SYNE1 pulmonary markers according to the invention described. The marker is not detected during the analysis of the genomic DNA from PBMCs

The specificity of the test was also verified in the presence of plasma DNA of healthy subjects. 10 plasma DNA samples were extracted from 1.5 mL of plasma of healthy subjects via the Qiagen QIAamp Circulating Nucleic Acid Kit (50) Cat. No./ID: 55104. The samples were subsequently subjected to a bisulfite conversion step by the Zymo Research EZ DNA Methylation-Lightning Kit (Reference D5031) then an analysis by Naica System digital PCR of the SYNE1 pulmonary markers according to the invention described. The markers are not detected during the analysis in the genomic DNA of healthy subjects.

Finally, the specificity of the test was determined in the presence of genomic DNA of other tissues. N=27 healthy tissue samples were collected (Transfer of biological samples MTA No. CT 69HCL22_0234 from the anatomopathology department of the Hospices Civiles de Lyon Sud et Est and of Hopital Lariboisière). For each sample, three 8 μm-thick tissue shavings prepared in paraffin were transferred. The genomic DNA of the brain-derived tissue shavings were extracted via the Qiagen QIAamp DNA FFPE Advanced Kit (50) (Cat. No./ID: 56604). The samples were subsequently subjected to a bisulfite conversion step by the Zymo Research EZ DNA Methylation-Lightning Kit (Reference D5031) then an analysis by Naica System digital PCR of the SYNE1 pulmonary markers according to the invention described. The negative test results for the SYNE1 epigenetic marker show that it was not detected in genomic DNA from tissue sections other than the lung confirming the absence of biological noise.

Positive control: A positive control is performed on genomic DNA samples from tissue sections of healthy lung tissues, by comparison with histological marking. 18 healthy lung tissue samples (1 white slide and three 8 μm-thick shavings prepared in paraffin) were collected from the anatomopathology department of the Hospices Civils de Lyon Sud et Est. For each sample, marking by an antibody anti-TTF1 was carried out on the white slides (antibody TTF1, Reference: DB089-0.5 Rabbit Monoclonal Anti-TTF-1 Clone (G21-G), DB Biotech) and each cell marked TTF1 (=pulmonary pneumocytes) was counted by means of the “HALO® image analysis” software. The samples were marked with the antibody TTF1. At the same time, the genomic DNA from the 8 μm-thick tissue shavings prepared in paraffin were extracted via the Qiagen QIAamp DNA FFPE Advanced Kit (50) (Cat. No./ID: 56604). The samples were subsequently subjected to a bisulfite conversion step by the Zymo Research EZ DNA Methylation-Lightning Kit (Reference D5031) then an analysis by Naica System digital PCR of the SYNE1 pulmonary markers according to the invention described.

The results show that the SYNE1 hypermethylated target is detected and quantified only in genomic DNA from healthy lung sections confirming the pulmonary specificity. The correlation between the TTF1 histological marking (=pulmonary pneumocytes) and the lung test described is 0.6 (R-Pearson coefficient) demonstrating the pulmonary and pneumocytic specificity of the analysis (FIG. 6).

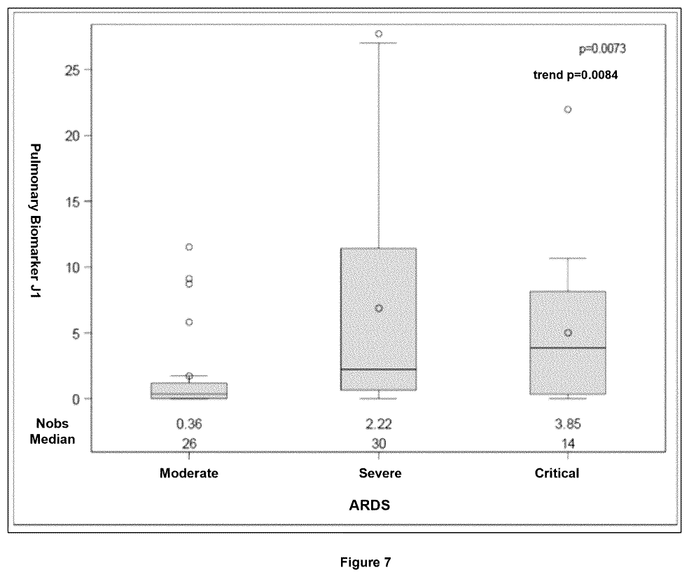

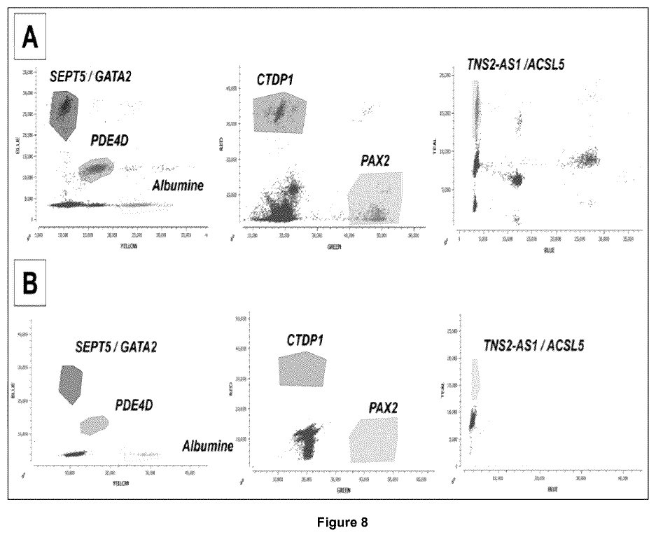

In a clinical application, a test according to the invention makes it possible to stratify patients with acute respiratory distress syndrome (ARDS) according to a severity index designated as low, severe or critical (Berlin classification). The results show that the SYNE1 hypermethylated target makes it possible to discriminate each subgroup of patients (moderate, severe, critical ARDS). A significant difference was observed between these 3 groups (p-value=0.0073, Jonckheere-Terpstra test). (FIG. 7)

Moreover, the results of the test according to the invention make it possible to monitor the pulmonary state of an ARDS patient with correlation indicators between the quantitative result of the SYNE1 hypermethylated target and the ventilatory markers 02 max/FiO2/PAO2 (respectively Pearson correlation coefficient of 0.44, 0.29 and 0.29).

### Example 5: Validation of a Second Biomarker Group for Kidney Degradation in the Treatment of the Kidney Transplant Patient

Two types of diagnostic tests targeting the kidney transplant rejection and the kidney lesion typology (total, vascular, and epithelial) were developed by selecting from the markers identified by the inventors.

According to prototype 1, the markers SEPT5 (glomerular endothelial marker); PDE4D/PAX2/ACSL5 (tubular epithelial marker); CTDP1 (total kidney marker) are tested on urine samples.

According to prototype 2, the markers GATA2 (renal endothelial marker); TNS2-AS1 (renal capillary endothelial marker)/PDE4D/PAX2 (tubular epithelial marker); CTDP1 (total kidney marker) are tested on plasma samples.

For the 2 prototypes, the “Albumin” marker is used as an ubiquitous marker quantifying all of the genomes present in the sample. The biological samples were obtained through hospital partners of the Necker nephrology departments and of hôpital Tenon-Pitié-Salpêtrière.

The concentrations of the various biomarkers associated in multiplex (here on a PCR mix) making up the kit dedicated to monitoring the status of kidney tissue damage in the context of kidney transplantation at the level of the total organ, the epithelial fraction thereof and the vascular fraction thereof are used according to the concentrations given in Table 16 below.

TABLE 16

| | Concentration |
|---|---|
| | in the reaction |
| PCR reagents | mixture (μM) |
| Albumin SENSE primer (internal control) | 0.5 |
| Albumin ANTISENSE primer (internal control) | 0.5 |
| Albumin PROBES (internal control) - ROX | 0.4 |
| GATA2 SENSE primer | 0.6 |
| GATA2 ANTISENSE primer | 0.6 |
| GATA2* PROBES - FAM | 0.55 |
| TNS2-AS1 SENSE primer | 0.6 |
| TNS2-AS1 ANTISENSE primer | 0.6 |
| TNS2-AS1 PROBES - YY | 0.55 |
| PDE4D SENSE primer | 0.5 |
| PDE4D ANTISENSE primer | 0.5 |
| PDE4D PROBES - FAM | 0.45 |
| PDE4D PROBES - ROX | 0.45 |
| PAX2 SENSE primer | 0.6 |
| PAX2 ANTISENSE primer | 0.6 |
| PAX2 - ATTO550 PROBES | 0.5 |
| CTDP1 SENSE primer | 0.6 |
| CTDP1 ANTISENSE primer | 0.6 |
| CTDP1 - Cy5 PROBES | 0.4 |
| H20 | |
| Total | |

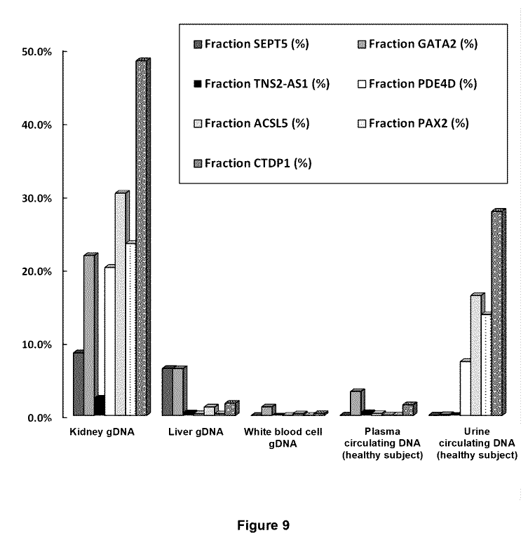

After validating these 2 prototypes at the metrological level (precision, repeatability, reproducibility), the 2 prototypes were tested on various so-called “positive” biological controls (genetic DNA from the kidney) and so-called “negative” controls (genetic DNA from the liver, white blood cells and plasma and urine circulating DNA of healthy subjects) (FIG. 9). The genomic DNA of the liver as well as white blood cells are controls assessed in the design of this test and of this methodology because they are contributors to the urine and plasma circulating DNA in the healthy subject.

These analyses made it possible to confirm the absence of detection or the very low-noise detection of our biomarkers in biological samples of negative controls: N=20 genomic DNA samples extracted from cell pellets of healthy subjects, N=30 plasma DNA samples of healthy subjects, N=15 urine DNA samples of healthy subjects, N=5 genomic DNA samples extracted from tissue sections (5 liver gDNA).

These gDNAs were mentioned as potential low-noise contributors to circulating DNA in healthy subjects: urine cf-DNA and plasma cf-DNA 5, respectively.

### Results:

The markers mentioned above are detected in 100% of the genomic DNA extracted from kidney tissue sections (N=22).

The markers proposed in this in vitro diagnostic test prototype make it possible to detect the renal DNA, specifically with very low biological noise in the genomic DNA of the liver and of the white blood cells (main contributors to circulating DNA) (FIG. 8). In the plasma circulating DNA of healthy subjects, there is very low biological background noise (slightly higher for the GATA2 endothelial marker (consistent with the literature)). As regards the urine circulating DNA of healthy subjects, the pan-kidney and renal epithelial markers are found, probably corresponding to the renal fraction physiologically present in the urine, in accordance with the literature.

The CTDP1 “total kidney” marker makes it possible to quantify the endothelial and epithelial fractions of the kidney. It is found in higher amounts in kidney genomic DNA compared to markers targeting only the epithelial and endothelial fraction. Similarly, the markers targeting the endothelial fraction of the kidney (GATA2) are found in greater amounts than the TNS2-AS1 and SEPT5 markers respectively targeting smaller vascular fractions (restricted respectively to the glomerulus and to the tubule).

### Example 6: Validation of the Renal Epithelial Marker Specificity: Comparison of the Molecular Markers (PAX2/ACSL5) and of the Histological Marker of the CD10 Renal Epithelium

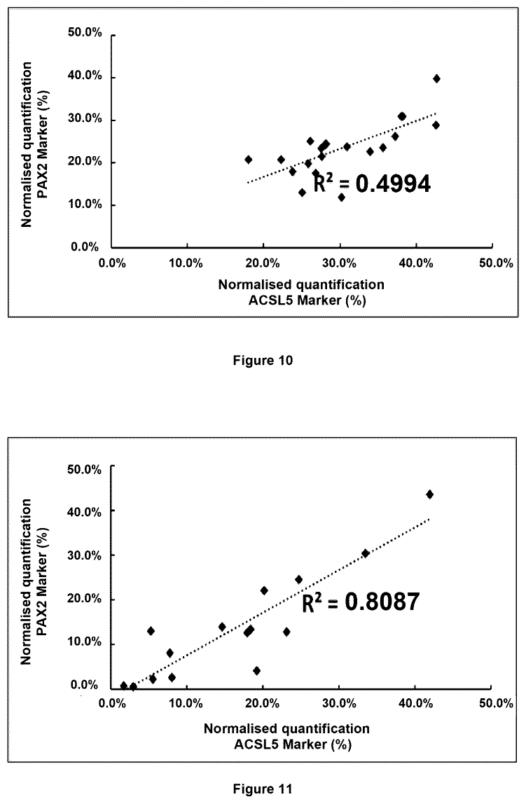

The inventors showed that the PAX2 and ACSL5 markers specifically target the renal epithelium. These markers were therefore tested to quantify the renal epithelium. In the total kidney gDNA (n=22) and in the urine plasma circulating DNA, these markers showed a good correlation of respectively R2=0.5 and R2=0.8, as indicated in FIG. 10 and FIG. 11.

The publication by Erger et al. showed that a fraction varying from 20 to 50% of the total urine circulating DNA in the healthy subject has a renal origin (Erger et al. Genome Medicine, (2020), doi.org/10.1186/s13073-020-00750-5) by proposing a bioinformatic deconvolution model (cfNOMe) of urine circulating DNA by NGS sequencing. This proportion is confirmed by the present result, suggesting a good specificity of the markers in terms of detection of circulating DNA of renal origin. Moreover, the urine circulating DNA of renal origin is rather of epithelial origin (absence of detection of vascular marker signals, e.g.: GATA2, TNS2-AS1 and SEPT-5 and detection of the markers PAX2, ACSL5 and PDE4D (R2 correlation (PAX2/ACSL5)=0.80 for the n=15 urine circulating DNA samples extracted from healthy subjects).

The results show that the PAX2 & ACSL5 markers specifically target the renal epithelium. In order to reinforce the results obtained, comparisons were carried out between the molecular data of the PAX2 and ACSL5 markers and the histological data (marking of renal epithelial cells on kidney white slides by the anti-CD10 antibody). For the n=22 healthy kidney genomic DNA for which PAX2 and ACSL5 were quantified, an immunohistochemical making of the renal epithelial cells via the anti-CD10 antibody on a paired white slide was carried out. CD10 is a surface marker specifically targeting the epithelial cells within a kidney section. Histological and molecular data show that the proportion of epithelial cells on these slides and kidney-paired gDNA varies between 11 and 40% and that there is no significant difference between the molecular markers and the histological markers targeting the renal epithelium (FIGS. 12A and 12B).

### Example 7: Analysis on Urine Samples of Kidney Transplant Patients

In order to validate the clinical relevance of the first prototype, a clinical collaboration in partnership with hôpital Necker and Tenon-Pitié Salpêtrière-nephrology/transplantation department made it possible to analyse N=50 urine samples of kidney transplant patients with/without transplant rejection, confirmed by anatomopathological analysis of the solid biopsy, and n=55 plasma samples of kidney transplant patients having biopsies with and without cell lesions, respectively, confirmed by anatomopathological analysis. The aim of these biomarkers is to specifically detect the cellular origin of the renal lysis, and thus the type of rejection in accordance with the classification of the kidney transplant rejection according to the Banff classification.

50 biological samples (2 ml of urine samples) from kidney transplant patients, having or not having kidney transplant rejection (rejection confirmed by solid biopsy), were analysed. Among these patients, 20 had T-cell mediated rejection (CTMR) and had no transplant rejection.

2 mL of urine were collected on the day of the kidney biopsy. Circulating DNA was extracted and the set of biomarkers (SEPT-5, ACSL5, PAX2, PDE4D, CTDP1) was analysed, simultaneously, by multiplexed digital PCR (6 colours, one for each marker and the albumin internal control).

A significant increase in the amount of biomarkers was observed in the urine of patients with T-cell rejection (Wilcoxon test, p-value<0.001). A regression model applied to the biomarkers showed good sensitivity and specificity of the diagnostic test of respectively 0.83 and 0.85, with an AUC score of 0.88.

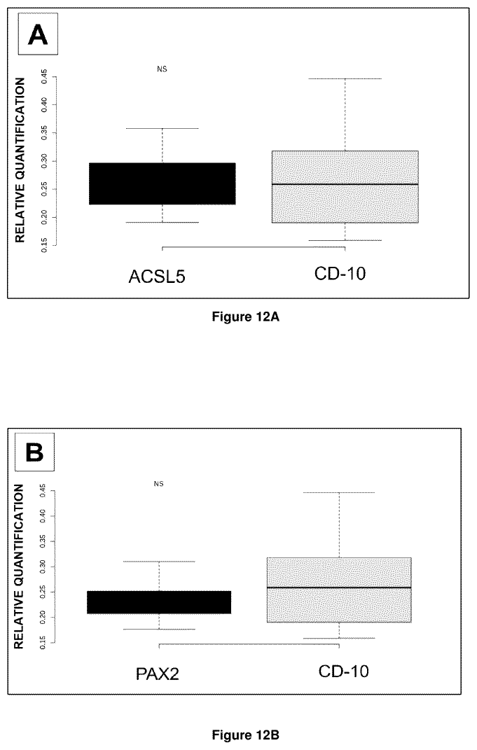

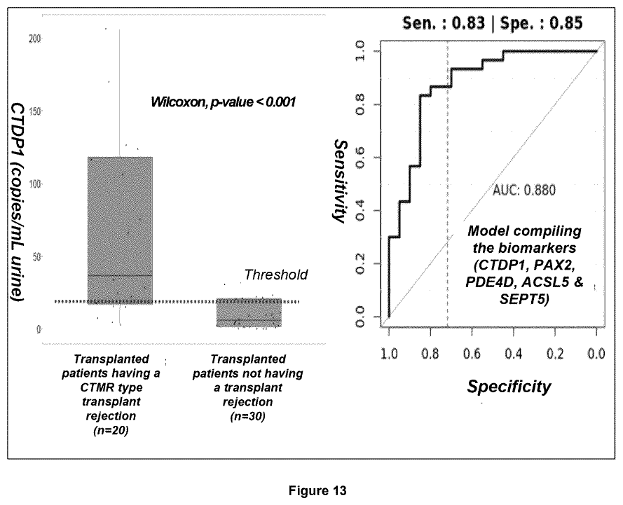

FIG. 13 shows the significant difference in quantification of the CTDP1 biomarker in urine between patients having a transplant rejection and those not having kidney transplant rejection (left graph). Logistic regression model compiling quantitative data from the biomarkers CTDP1, PAX2, PDE4D, ACSL5, and SEPT5 to predict the presence or the absence of transplant rejection (right graph).

### Example 8: Analysis on Plasma Samples of Kidney Transplant Patients

55 biological samples of kidney transplant patients, having or not having kidney transplant rejection (rejection confirmed by solid biopsy with biopsies having glomerular (g), tubular (t), vascular (v) and capillary (ptc) type graft lesions) were analysed.

0.6-1.5 mL of plasma were collected on the day of the kidney biopsy. Circulating DNA was extracted and the set of biomarkers (GATA2, TNS2-AS1, PAX2, PDE4D, CTDP1) was analysed, simultaneously, by multiplex digital PCR (6 colours, one for each marker+albumin internal control).

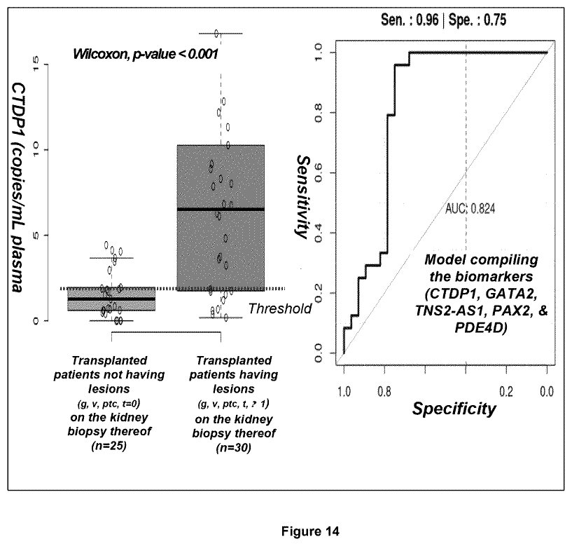

A significant increase in the amount of biomarkers was also observed in the plasmas of transplant patients having a degradation of the kidney graft. A regression model applied to the biomarkers showed good sensitivity and specificity of the diagnostic test of respectively 0.96 and 0.75, with an AUC score of 0.82. These results highlight the reliability of the test in the analysis of transplant rejection in plasma samples. A significant increase in the amount of vascular biomarkers (specifically the GATA2 and TNS2-AS1 markers) was also observed in the plasmas of patients having vascular lesions of the kidney graft as expected (FIG. 14).

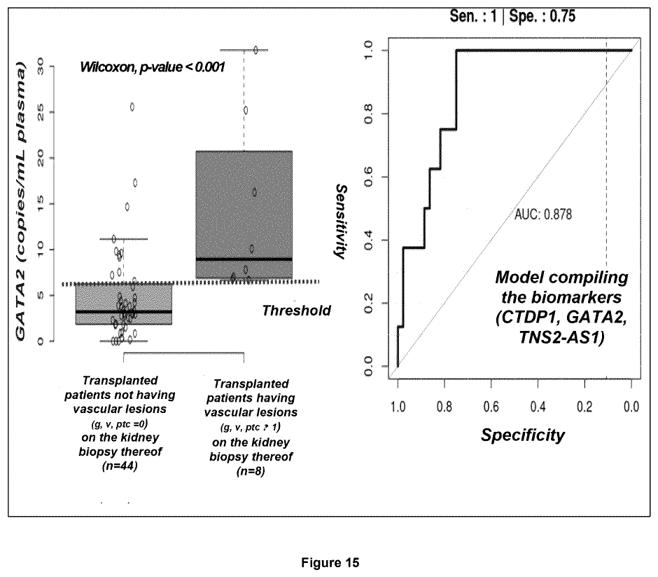

A regression model applied to the “total kidney” and “vascular” biomarkers (specifically the markers CTDP1, GATA2 and TNS2-AS1) showed good sensitivity and specificity of the diagnostic test, with an AUC of 0.878 (FIG. 15).

Together, these results demonstrate:
the technical reliability of the developed test in the detection of biomarkers of interest in the urine and plasma of patients,
the sensitivity and technical specificity of the developed test,
the clinical interest of the developed test in detecting kidney transplant rejection,
the clinical interest of the developed test in the characterisation of epithelial and/or vascular lesions of the kidney graft.

1-18. (canceled)

19. A kit for detecting in a biological sample a methylated cell-free DNA target nucleotide sequence, said nucleotide sequence being specific to a cell type of an organ selected from: a brain, a lung and a kidney, said kit comprising:
i) for detecting a specific cell-free DNA target sequence of a type of cells present in the brain, at least: one sense primer comprising a nucleotide sequence SEQ ID No. 1, or a nucleotide sequence having at least 80% identity with SEQ ID No. 1 and one antisense primer, comprising a nucleotide sequence SEQ ID No. 2, or a nucleotide sequence having at least 80% identity with SEQ ID No. 2, and/or
one sense primer comprising a nucleotide sequence SEQ ID No. 4, or a nucleotide sequence having at least 80% identity with SEQ ID No. 4 and one antisense primer, comprising a nucleotide sequence SEQ ID No. 5, or a nucleotide sequence having at least 80% identity with SEQ ID No. 5;
ii) for detecting a specific cell-free DNA target sequence of a type of cells present in the lung, at least: one sense primer, comprising a nucleotide sequence SEQ ID No. 7, or a nucleotide sequence having at least 80% identity with SEQ ID No. 7 and one antisense primer, comprising a nucleotide sequence SEQ ID No. 8, or a nucleotide sequence having at least 80% identity with SEQ ID No. 8, and
iii) for detecting a specific cell-free DNA target sequence of a type of cells present in the kidney, at least:
one sense primer comprising a sequence SEQ ID No. 10, or a sequence having at least 80% identity with SEQ ID No. 10 and one antisense primer, comprising a sequence SEQ ID No. 11, or a sequence having at least 80% identity with SEQ ID No. 11, and/or
one sense primer comprising a sequence SEQ ID No. 13, or a sequence having at least 80% identity with SEQ ID No. 13 and one antisense primer, comprising a sequence SEQ ID No. 14, or a sequence having at least 80% identity with SEQ ID No. 14, and/or
one sense primer comprising a sequence SEQ ID No. 19, or a sequence having at least 80% identity with SEQ ID No. 19 and one antisense primer, comprising a sequence SEQ ID No. 20, or a sequence having at least 80% identity with SEQ ID No. 20, and/or
one sense primer comprising a sequence SEQ ID No. 22, or a sequence having at least 80% identity with SEQ ID No. 22 and one antisense primer, comprising a sequence SEQ ID No. 23, or a sequence having at least 80% identity with SEQ ID No. 23, and/or
one sense primer comprising a sequence SEQ ID No. 25, or a sequence having at least 80% identity with SEQ ID No. 25 and one antisense primer, comprising a sequence SEQ ID No. 26, or a sequence having at least 80% identity with SEQ ID No. 26, and/or
one sense primer comprising a sequence SEQ ID No. 28, or a sequence having at least 80% identity with SEQ ID No. 28 and one antisense primer, comprising a sequence SEQ ID No. 29, or a sequence having at least 80% identity with SEQ ID No. 29, and/or
one sense primer comprising a sequence SEQ ID No. 31, or a sequence having at least 80% identity with SEQ ID No. 31 and one antisense primer, comprising a sequence SEQ ID No. 32, or a sequence having at least 80% identity with SEQ ID No. 32, and/or
one sense primer comprising a sequence SEQ ID No. 34, or a sequence having at least 80% identity with SEQ ID No. 34 and one antisense primer, comprising a sequence SEQ ID No. 35, or a sequence having at least 80% identity with SEQ ID No. 35, and/or
one sense primer comprising a sequence SEQ ID No. 37, or a sequence having at least 80% identity with SEQ ID No. 37 and one antisense primer, comprising a sequence SEQ ID No. 38, or a sequence having at least 80% identity with SEQ ID No. 38, and/or
one sense primer comprising a sequence SEQ ID No. 40, or a sequence having at least 80% identity with SEQ ID No. 40 and one antisense primer, comprising a sequence SEQ ID No. 41, or a sequence having at least 80% identity with SEQ ID No. 41, and/or
one sense primer comprising a sequence SEQ ID No. 43, or a sequence having at least 80% identity with SEQ ID No. 43 and one antisense primer, comprising a sequence SEQ ID No. 44, or a sequence having at least 80% identity with SEQ ID No. 44, and/or
one sense primer comprising a sequence SEQ ID No. 46, or a sequence having at least 80% identity with SEQ ID No. 46 and one antisense primer, comprising a sequence SEQ ID No. 47, or a sequence having at least 80% identity with SEQ ID No. 47, and/or
one sense primer comprising a sequence SEQ ID No. 49, or a sequence having at least 80% identity with SEQ ID No. 49 and one antisense primer, comprising a sequence SEQ ID No. 50, or a sequence having at least 80% identity with SEQ ID No. 50, and/or
one sense primer comprising a sequence SEQ ID No. 52, or a sequence having at least 80% identity with SEQ ID No. 52 and one antisense primer, comprising a sequence SEQ ID No. 53, or a sequence having at least 80% identity with SEQ ID No. 53.

20. The kit according to claim 19, further comprising a probe comprising:
i) for detecting a specific cell-free DNA target sequence of a type of cells present in the brain, at least one nucleotide sequence selected from: SEQ ID No. 3, SEQ ID No. 6 and a nucleotide sequence having at least 80% identity with SEQ ID No. 3 or SEQ ID No. 6,
ii) for detecting a specific cell-free DNA target sequence of a type of cells present in the lung, at least one nucleotide sequence selected from: SEQ ID No. 9 and a nucleotide sequence having at least 80% identity with SEQ ID No. 9, and
iii) for detecting a specific cell-free DNA target sequence of a type of cells present in the kidney, at least one nucleotide sequence selected from: SEQ ID No. 12, SEQ ID No. 15, SEQ ID No. 21, SEQ ID No. 24, SEQ ID No. 27, SEQ ID No. 30, SEQ ID No. 33, SEQ ID No. 36, SEQ ID No. 39, SEQ ID No. 42, SEQ ID No. 45, SEQ ID No. 48, SEQ ID No. 51, SEQ ID No. 54, and a nucleotide sequence having at least 80% identity with SEQ ID No. 12, SEQ ID No. 15, SEQ ID No. 21, SEQ ID No. 24, SEQ ID No. 27, SEQ ID No. 30, SEQ ID No. 33, SEQ ID No. 36, SEQ ID No. 39, SEQ ID No. 42, SEQ ID No. 45, SEQ ID No. 48, SEQ ID No. 51 or SEQ ID No. 54.

21. The kit according to claim 19, for detecting in a biological sample a specific cell-free DNA target sequence of a type of cells present in the brain.

22. The kit according to claim 19, for detecting in a biological sample a specific cell-free DNA target sequence of a type of cells present in the lung.

23. The kit according to claim 19, for detecting in a biological sample a specific cell-free DNA target sequence of a type of cells present in the kidney.

24. A method for identifying a specific cell-free DNA target sequence of a type of cells present in a first organ selected from: the lung, the brain, and the kidney, the method comprising at least the following steps of:
a) determining, in at least one genomic DNA methylation database, the position and the degree of methylation of the CpG islands of specific sequences of said cell type of said organ,
b) identifying, in the genomic DNA of said cell type of said healthy organ, specifically hypermethylated CpG islands,
c) comparing the degree of methylation of the hypermethylated CpG islands of said cell type of said first organ with the degree of methylation of the same CpG islands of a second cell type, different from the first cell type,
d) selecting the CpG islands only hypermethylated in the genomic DNA of said cell type of said first organ after comparison performed in step c),
e) comparing the degree of methylation of the hypermethylated CpG islands of said cell type of said first organ with the degree of methylation of the same CpG islands of the genomic DNA of the other organs of said first organ,
f) comparing the degree of methylation of the hypermethylated CpG islands of said cell type of said first organ with the degree of methylation of the same CpG islands of the genomic DNA of the healthy white blood cells,
g) comparing the degree of methylation of the hypermethylated CpG islands of said cell type of said first organ with the degree of methylation of the same CpG islands of the cell-free DNA of all biological matrices combined from healthy subjects,
h) selecting the CpG islands only hypermethylated in the genomic DNA of said cell type of said first organ after comparison performed in step e, f and g), and
i) identifying one or more target hypermethylated sequences of specific cell-free DNA of said cell type of the first organ from the comparison performed in step h).

25. A method for detecting in a biological sample a target sequence of cell-free DNA, said sequence being specific to a type of cells present in an organ selected from: the lung, the brain and the kidney, said method comprising at least the following steps of:
a) bringing together, under conditions suitable for an amplification of the nucleic acids, the cell-free DNA previously extracted from a biological sample and a pair of primers consisting of a sense primer and an antisense primer, each of the primers comprising:
a sense primer and an antisense primer likely to hybridise with a nucleotide sequence selected from: SEQ ID No. 55, SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70 and SEQ ID No. 71,
b) amplifying the reaction of said target sequence,
c) detecting the presence of the amplified sequence during step b).

26. The method for detecting in a biological sample a target sequence of cell-free DNA according to claim 25, said sequence being specific to a type of cells present in an organ selected from: the lung, the brain and the kidney, said method comprising at least the following steps of:
a) bringing together, under conditions suitable for an amplification of the nucleic acids, the cell-free DNA previously extracted from a biological sample and a pair of primers consisting of a sense primer and an antisense primer, each of the primers comprising:
i) for detecting a specific target sequence of a type of cells present in the brain, at least: one sense primer comprising a sequence SEQ ID No. 1, or a sequence having at least 80% identity with SEQ ID No. 1 and one antisense primer, comprising a sequence SEQ ID No. 2, or a sequence having at least 80% identity with SEQ ID No. 2, and/or
a sense primer comprising a sequence SEQ ID No. 4, or a sequence having at least 80% identity with SEQ ID No. 4 and an antisense primer comprising a sequence SEQ ID No. 5, or a sequence having at least 80% identity with SEQ ID No. 5,
ii) for detecting a specific target sequence of a type of cells present in the lung, at least: one sense primer comprising a sequence SEQ ID No. 7, or a sequence having at least 80% identity with SEQ ID No. 7 and one antisense primer comprising a sequence SEQ ID No. 8, or a sequence having at least 80% identity with SEQ ID No. 8, and
iii) for detecting a specific target sequence of a type of cells present in the kidney, at least:
one sense primer comprising a sequence SEQ ID No. 10, or a sequence having at least 80% identity with SEQ ID No. 10 and one antisense primer, comprising a sequence SEQ ID No. 11, or a sequence having at least 80% identity with SEQ ID No. 11, and/or
one sense primer comprising a sequence SEQ ID No. 13, or a sequence having at least 80% identity with SEQ ID No. 13 and one antisense primer, comprising a sequence SEQ ID No. 14, or a sequence having at least 80% identity with SEQ ID No. 14, and/or
one sense primer comprising a sequence SEQ ID No. 19, or a sequence having at least 80% identity with SEQ ID No. 19 and one antisense primer, comprising a sequence SEQ ID No. 20, or a sequence having at least 80% identity with SEQ ID No. 20, and/or
one sense primer comprising a sequence SEQ ID No. 22, or a sequence having at least 80% identity with SEQ ID No. 22 and one antisense primer, comprising a sequence SEQ ID No. 23, or a sequence having at least 80% identity with SEQ ID No. 23, and/or
one sense primer comprising a sequence SEQ ID No. 25, or a sequence having at least 80% identity with SEQ ID No. 25 and one antisense primer, comprising a sequence SEQ ID No. 26, or a sequence having at least 80% identity with SEQ ID No. 26, and/or
one sense primer comprising a sequence SEQ ID No. 28, or a sequence having at least 80% identity with SEQ ID No. 28 and one antisense primer, comprising a sequence SEQ ID No. 29, or a sequence having at least 80% identity with SEQ ID No. 29, and/or
one sense primer comprising a sequence SEQ ID No. 31, or a sequence having at least 80% identity with SEQ ID No. 31 and one antisense primer, comprising a sequence SEQ ID No. 32, or a sequence having at least 80% identity with SEQ ID No. 32, and/or
one sense primer comprising a sequence SEQ ID No. 34, or a sequence having at least 80% identity with SEQ ID No. 34 and one antisense primer, comprising a sequence SEQ ID No. 35, or a sequence having at least 80% identity with SEQ ID No. 35, and/or
one sense primer comprising a sequence SEQ ID No. 37, or a sequence having at least 80% identity with SEQ ID No. 37 and one antisense primer, comprising a sequence SEQ ID No. 38, or a sequence having at least 80% identity with SEQ ID No. 38, and/or
one sense primer comprising a sequence SEQ ID No. 40, or a sequence having at least 80% identity with SEQ ID No. 40 and one antisense primer, comprising a sequence SEQ ID No. 41, or a sequence having at least 80% identity with SEQ ID No. 41, and/or
one sense primer comprising a sequence SEQ ID No. 43, or a sequence having at least 80% identity with SEQ ID No. 43 and one antisense primer, comprising a sequence SEQ ID No. 44, or a sequence having at least 80% identity with SEQ ID No. 44, and/or
one sense primer comprising a sequence SEQ ID No. 46, or a sequence having at least 80% identity with SEQ ID No. 46 and one antisense primer, comprising a sequence SEQ ID No. 47, or a sequence having at least 80% identity with SEQ ID No. 47, and/or
one sense primer comprising a sequence SEQ ID No. 49, or a sequence having at least 80% identity with SEQ ID No. 49 and one antisense primer, comprising a sequence SEQ ID No. 50, or a sequence having at least 80% identity with SEQ ID No. 50, and/or
one sense primer comprising a sequence SEQ ID No. 52, or a sequence having at least 80% identity with SEQ ID No. 52 and one antisense primer, comprising a sequence SEQ ID No. 53, or a sequence having at least 80% identity with SEQ ID No. 53,
b) amplifying the reaction of said target sequence,
c) detecting the presence of the sequence amplified during step b).

27. The method for detecting in a biological sample a target sequence of cell-free DNA according to claim 25, wherein the target sequence is a specific cell-free DNA target sequence of a type of cells present in the brain.

28. The method for detecting in a biological sample a target sequence of cell-free DNA according to claim 25, wherein the target sequence is a specific cell-free DNA target sequence of a type of cells present in the lung.

29. The method for detecting in a biological sample a target sequence of cell-free DNA according to claim 25, wherein the target sequence is a specific cell-free DNA target sequence of a type of cells present in the kidney.

30. The method for detecting in a biological sample a target sequence of cell-free DNA according to claim 29, for detecting the type of renal cell that is degrading, said cell type being selected from the epithelial cells and the vascular cells.

31. The method for detecting in a biological sample a target sequence of cell-free DNA according to claim 25, for detecting kidney transplant rejection in a kidney transplant patient.

32. The method for detecting in a biological sample a target sequence of cell-free DNA according to claim 29, for in vitro diagnosis of the lesional typology of the rejection, said rejection being of the epithelial or vascular type, depending on the histological analyses of the solid biopsy of the kidney graft.

33. A method for specifically detecting the degradation of a brain and/or for in vitro diagnosis or monitoring the progression of a stroke, comprising detecting with the kit according claim 19 in a biological sample a methylated cell-free DNA target nucleotide sequence, said nucleotide sequence being specific to a cell type of the brain.

34. A method for specifically detecting the degradation of a lung and/or for in vitro diagnosis or monitoring the progression of a respiratory distress syndrome comprising detecting with the kit according claim 19 in a biological sample a methylated cell-free DNA target nucleotide sequence, said nucleotide sequence being specific to a cell type of the lung.

35. A method for specifically detecting the degradation of the kidney and/or for the in vitro diagnosis or monitoring the progression of a kidney injury or graft rejection, comprising detecting with the kit according claim 19 in a biological sample a methylated cell-free DNA target nucleotide sequence, said nucleotide sequence being specific to a cell type of the kidney.

36. A nucleotide sequence for the use thereof in the kit claim 19, selected from:
a sense primer comprising a nucleotide sequence selected from: SEQ ID No. 1, SEQ ID No. 4, SEQ ID No. 7, SEQ ID No. 10, SEQ ID No. 13, SEQ ID No. 19, SEQ ID No. 22, SEQ ID No. 25, SEQ ID No. 28, SEQ ID No. 31, SEQ ID No. 34, SEQ ID No. 37, SEQ ID No. 40, SEQ ID No. 43, SEQ ID No. 46, SEQ ID No. 49 and SEQ ID No. 52, or a nucleotide sequence having at least 80% identity with said sequences;
an antisense primer comprising a nucleotide sequence selected from: SEQ ID No. 2, SEQ ID No. 5, SEQ ID No. 8, SEQ ID No. 11, SEQ ID No. 14, SEQ ID No. 20, SEQ ID No. 23, SEQ ID No. 26, SEQ ID No. 29, SEQ ID No. 32, SEQ ID No. 35, SEQ ID No. 38, SEQ ID No. 41, SEQ ID No. 44, SEQ ID No. 47, SEQ ID No. 50 and SEQ ID No. 53, or a nucleotide sequence having at least 80% identity with said sequences; and
a probe comprising a nucleotide sequence selected from: SEQ ID No. 3, SEQ ID No. 6, SEQ ID No. 9, SEQ ID No. 12, SEQ ID No. 15, SEQ ID No. 21, SEQ ID No. 24, SEQ ID No. 27, SEQ ID No. 30, SEQ ID No. 33, SEQ ID No. 36, SEQ ID No. 39, SEQ ID No. 42, SEQ ID No. 45, SEQ ID No. 48, SEQ ID No. 51 and SEQ ID No. 54, or a nucleotide sequence having at least 80% identity with said sequences.

37. The kit according to claim 20, detecting in a biological sample a specific cell-free DNA target sequence of a type of cells present in the brain, or in the lung, or in the kidney.

38. The method for detecting in a biological sample a target sequence of cell-free DNA according to claim 26, for detecting in a biological sample a specific cell-free DNA target sequence of a type of cells present in the brain or in the kidney.


SEQUENCE LISTING (71 sequences)

SEQ ID NO: 1 - DNA, 19 bp, synthetic construct
  source 1..19: mol_type = other DNA
tcgggttttcgggtaacgt

SEQ ID NO: 2 - DNA, 17 bp, synthetic construct
  source 1..17: mol_type = other DNA
cttcgccacgcaacaac

SEQ ID NO: 3 - DNA, 17 bp, synthetic construct
  source 1..17: mol_type = other DNA
tgaagagttggtagcgt

SEQ ID NO: 4 - DNA, 18 bp, synthetic construct
  source 1..18: mol_type = other DNA
cgtagggtcggaaggagg

SEQ ID NO: 5 - DNA, 18 bp, synthetic construct
  source 1..18: mol_type = other DNA
atccgaacgaacgacgaa

SEQ ID NO: 6 - DNA, 21 bp, synthetic construct
  source 1..21: mol_type = other DNA
cgtttaaggttgtattagttg

SEQ ID NO: 7 - DNA, 19 bp, synthetic construct
  source 1..19: mol_type = other DNA
gttagatcgtaggttgcgc

SEQ ID NO: 8 - DNA, 18 bp, synthetic construct
  source 1..18: mol_type = other DNA
acgcgaactcgaacactt

SEQ ID NO: 9 - DNA, 15 bp, synthetic construct
  source 1..15: mol_type = other DNA
cgcggcgtaggtata

SEQ ID NO: 10 - DNA, 18 bp, synthetic construct
  source 1..18: mol_type = other DNA
ggttttagcggatggcga

SEQ ID NO: 11 - DNA, 20 bp, synthetic construct
  source 1..20: mol_type = other DNA
gctcctctacgaaacgaaca

SEQ ID NO: 12 - DNA, 13 bp, synthetic construct
  source 1..13: mol_type = other DNA
tggcgagcgcgtt

SEQ ID NO: 13 - DNA, 24 bp, synthetic construct
  source 1..24: mol_type = other DNA
ggcgaaattcggtgtatataattt

SEQ ID NO: 14 - DNA, 22 bp, synthetic construct
  source 1..22: mol_type = other DNA
gaacgacaaaaactctacgaaa

SEQ ID NO: 15 - DNA, 17 bp, synthetic construct
  source 1..17: mol_type = other DNA
tcgtttcgtttcgttcg

SEQ ID NO: 16 - DNA, 22 bp, synthetic construct
  source 1..22: mol_type = other DNA
gggatggaaagaattttatgtt

SEQ ID NO: 17 - DNA, 22 bp, synthetic construct
  source 1..22: mol_type = other DNA
aaacaaactaaccccaaattct

SEQ ID NO: 18 - DNA, 17 bp, synthetic construct
  source 1..17: mol_type = other DNA
agggtttttataattta

SEQ ID NO: 19 - DNA, 23 bp, synthetic construct
  source 1..23: mol_type = other DNA
tcgggttaggttttgttaaggta

SEQ ID NO: 20 - DNA, 20 bp, synthetic construct
  source 1..20: mol_type = other DNA
tcctccgaaacgaaacgaaa

SEQ ID NO: 21 - DNA, 18 bp, synthetic construct
  source 1..18: mol_type = other DNA
tggcgtttttgtgcgcgt

SEQ ID NO: 22 - DNA, 24 bp, synthetic construct
  source 1..24: mol_type = other DNA
tttaaatcggtgagtaggataggt

SEQ ID NO: 23 - DNA, 27 bp, synthetic construct
  source 1..27: mol_type = other DNA
attccaaaaaccgaatcaaaaatatca

SEQ ID NO: 24 - DNA, 17 bp, synthetic construct
  source 1..17: mol_type = other DNA
agtttggcgtgtttgag

SEQ ID NO: 25 - DNA, 21 bp, synthetic construct
  source 1..21: mol_type = other DNA
gcggttgtgtttgagatagag

SEQ ID NO: 26 - DNA, 21 bp, synthetic construct
  source 1..21: mol_type = other DNA
caaaactaacgaccgaaaacc

SEQ ID NO: 27 - DNA, 19 bp, synthetic construct
  source 1..19: mol_type = other DNA
tgttaagagagtttcgagt

SEQ ID NO: 28 - DNA, 47 bp, synthetic construct
  source 1..47: mol_type = other DNA
ggatattttgggtcggttttttgttgggtcggttttttgtttgtatg

SEQ ID NO: 29 - DNA, 41 bp, synthetic construct
  source 1..41: mol_type = other DNA
ctcgctcgtaattcctccaaatccctcgctcgtaattcctc

SEQ ID NO: 30 - DNA, 42 bp, synthetic construct
  source 1..42: mol_type = other DNA
tgtatggacgttttgaagttacgttttgaagttattttgttt

SEQ ID NO: 31 - DNA, 45 bp, synthetic construct
  source 1..45: mol_type = other DNA
gaaacggtgagatgttcggagggtttaaatgaaacggtgagatgt

SEQ ID NO: 32 - DNA, 51 bp, synthetic construct
  source 1..51: mol_type = other DNA
ctttactattacgaaacgttcctaatacacgaaacgttcctaatacaacaa

SEQ ID NO: 33 - DNA, 32 bp, synthetic construct
  source 1..32: mol_type = other DNA
aagcgtcggttgtttgtcggaggaagcgtcgg

SEQ ID NO: 34 - DNA, 27 bp, synthetic construct
  source 1..27: mol_type = other DNA
ttggttttcgaattttgtttgtttata

SEQ ID NO: 35 - DNA, 26 bp, synthetic construct
  source 1..26: mol_type = other DNA
ctaataataaactccaaatctacgca

SEQ ID NO: 36 - DNA, 19 bp, synthetic construct
  source 1..19: mol_type = other DNA
acgaattttagtacggggt

SEQ ID NO: 37 - DNA, 17 bp, synthetic construct
  source 1..17: mol_type = other DNA
tgggggagggaaggtcg

SEQ ID NO: 38 - DNA, 56 bp, synthetic construct
  source 1..56: mol_type = other DNA
ctataaaaaacaaataaactaaaaaacacgacaaataaactaaaaaacacgatcga

SEQ ID NO: 39 - DNA, 32 bp, synthetic construct
  source 1..32: mol_type = other DNA
tgcggttttggtatcgaacgtgcggttttggt

SEQ ID NO: 40 - DNA, 32 bp, synthetic construct
  source 1..32: mol_type = other DNA
agtgtgggtgggcgcgcgcggggggagttggt

SEQ ID NO: 41 - DNA, 47 bp, synthetic construct
  source 1..47: mol_type = other DNA
tcgacaactttaacgacctaacttatctctctcgacaactttaacga

SEQ ID NO: 42 - DNA, 43 bp, synthetic construct
  source 1..43: mol_type = other DNA
agttggttttacgtttagaagttacgtttagaaggaaagttag

SEQ ID NO: 43 - DNA, 41 bp, synthetic construct
  source 1..41: mol_type = other DNA
cgtttgttgttttatttggtcgatggtcgaggacgagttgc

SEQ ID NO: 44 - DNA, 39 bp, synthetic construct
  source 1..39: mol_type = other DNA
acaaaacgaaccgaaaacgccgcgtacaacaacccaaaa

SEQ ID NO: 45 - DNA, 33 bp, synthetic construct
  source 1..33: mol_type = other DNA
acgagttgcgcgtcgtcgtcgtttgcgttttcg

SEQ ID NO: 46 - DNA, 41 bp, synthetic construct
  source 1..41: mol_type = other DNA
agttattttcgggtgtttcgttcgggtgtttcgtttttgcg

SEQ ID NO: 47 - DNA, 47 bp, synthetic construct
  source 1..47: mol_type = other DNA
cgaatccaaactaaaactcctaaaacgctccataaacgaatccaaac

SEQ ID NO: 48 - DNA, 40 bp, synthetic construct
  source 1..40: mol_type = other DNA
tgcgtttatgcgtaggtttgcgtaggttttttttttttag

SEQ ID NO: 49 - DNA, 25 bp, synthetic construct
  source 1..25: mol_type = other DNA
tcgtatttgtttttattttggttgt

SEQ ID NO: 50 - DNA, 21 bp, synthetic construct
  source 1..21: mol_type = other DNA
ctttctcccgaataaacaccg

SEQ ID NO: 51 - DNA, 20 bp, synthetic construct
  source 1..20: mol_type = other DNA
taaatttcggtttcggttcg

SEQ ID NO: 52 - DNA, 28 bp, synthetic construct
  source 1..28: mol_type = other DNA
atttatattaggtagtgagaagatgtgt

SEQ ID NO: 53 - DNA, 29 bp, synthetic construct
  source 1..29: mol_type = other DNA
aatataaataccaaaacaaaaacacacaa

SEQ ID NO: 54 - DNA, 20 bp, synthetic construct
  source 1..20: mol_type = other DNA
tggttttgagttgatggtag

SEQ ID NO: 55 - DNA, 121 bp, Homo sapiens
  source 1..121: mol_type = genomic DNA
cccgagtcccagtccaggagcccggctctagcggatggcgagggggtggcgagcgcgcttgggtgcccgccccgcagaggagcgtctggtccacagccggatctcctcggtccccaacccg

SEQ ID NO: 56 - DNA, 221 bp, Homo sapiens
  source 1..221: mol_type = genomic DNA
atatgatggtctttgcaaaaaggaacaaaacaacacaaaagcccaccaggctgctgctttgtggaaagacggtgtgtgtcgtgtgaaggcgaaacccggtgtacataacccctccccctccgccccgccccgcccggccccgtagagtccctgtcgcccgccggccctgcctgtagatacgccccgctgtctgtgctgtgagagtcgccgctcgctggggg

SEQ ID NO: 57 - DNA, 170 bp, Homo sapiens
  source 1..170: mol_type = genomic DNA
cggacgtcgctccatccttcccgcctgacggatgcgtctgctgcgcaggcacaagctgtcgggccaggccctgccaaggcagaacagtggcgcttctgtgcgcgctgctccgctccgctccggaggatggggcccgcccgggaagccagggagtgatggagaaagcagac

SEQ ID NO: 58 - DNA, 127 bp, Homo sapiens
  source 1..127: mol_type = genomic DNA
accttagtatattgaatcttcaaatcggtgagtaggacaggtaagtctggcgtgcctgaggatgacatttctgattcggctcctggaacacaggtgactggctcagcaatttggttctgatagagca

SEQ ID NO: 59 - DNA, 494 bp, Homo sapiens
  source 1..494: mol_type = genomic DNA
cgagcacacagaggcagcggctgtgtttgagacagagtgtgtttgtcaagagagccccgagccaggccctcggccgccagccctggtgccggcgtcttcctgggcctccgggagccccggctgcacgtgagccgccgggagggctgtcgggaagccccctgggcacctggtcctccccaccggggcagcgcccgcctcatcaaagccactgtggctgctggcgccgggccagctgggccctggcgggcgaggggtgggtgggtgcccgccagcccctggcactgtgccccggcccgtgcccgtgccaggagcgtcgctgactcagccggaagagccttatctggcccctgcgccaccgcctggccgtcgggtcaccgccgcggggtcaccgccgccgcggcctggccccacgcccaccgccggcgtcccaccctggtggctgctgccaagcgccggccccgccccatgctttctaataacacacgcggcagccc

SEQ ID NO: 60 - DNA, 124 bp, Homo sapiens
  source 1..124: mol_type = genomic DNA
gaactggggacactctgggccggccttctgcctgcatggacgctctgaagccaccctgtctctggaggaaccacgagcgagggaagaaggacagggactcgtgtggcaggaagaactcagagcc

SEQ ID NO: 61 - DNA, 121 bp, Homo sapiens
  source 1..121: mol_type = genomic DNA
cggtgagatgctcggaggaagcgccggctgcttgctgcatcaggaacgtttcgtaatagcaaaggcactatcgggaagcgttacgtaagctgtccattaaggacactgattaggaacgttc

SEQ ID NO: 62 - DNA, 452 bp, Homo sapiens
  source 1..452: mol_type = genomic DNA
tggcaggctccccgcggaaccctgctgagacccggagacaatcgggccgtgcttctccctcctccacgaacagccacggtttatttggagcggccggggccggcggcctgacaactggtaaatccgtttcgttaggcacaatttgtctgcaatttgtcagcccggctgggaaacgctccccagacgcctcggctgccgcacgggccctacctggttctcgaatcctgcctgctcataaacgaatcctagcacggggtgcctgcgtagacctggagctcaccaccagatgttcccgacctcgggagaggaggctttttccaaaacaacgaatttccttccttgttttccggtaaaggagcgttcgccacacacggggtccctgaacgcggggcctttcccctcgtggttggagcaacgcggagttcaagcctggccgccacagaataatttta

SEQ ID NO: 63 - DNA, 201 bp, Homo sapiens
  source 1..201: mol_type = genomic DNA
ttgcaaacccctccgggctgcttattcctggtgccatgcgcagcccttgggacctagaagattgtgggggagggaaggtcgcacgtgcggttttggcaccgaccgtgcctcctagtccacctgtcccccacagctagctgccaactcggacggaagcgcggaacagcgcaatgcaaaccgcccaaagtgaaaggaatgtaa

SEQ ID NO: 64 - DNA, 152 bp, Homo sapiens
  source 1..152: mol_type = genomic DNA
cgggagcatggagtgagtgtgggtgggcgcgcggggggagctggccccacgctcagaaggaaagccaggtcgctaaagctgccgagagagacaccaggagcaccgggaacgccgggagcagggaccccgccgccccgttgccgctgccgcag

SEQ ID NO: 65 - DNA, 106 bp, Homo sapiens
  source 1..106: mol_type = genomic DNA
atctggccgaggacgagctgcgcgccgcttgcgctcccggcccgctctgctggggggcgctggcggcgcagcttgcgctgctgggccttgggctgctgcacgcgtt

SEQ ID NO: 66 - DNA, 123 bp, Homo sapiens
  source 1..123: mol_type = genomic DNA
cgtcacactagccacctccgggtgccccgcccctgcgttcatgcgtaggctcctcctccccaggagcttcagcctggacccgctcatggagcggcgctgggacttagacctcacctacgtgac

SEQ ID NO: 67 - DNA, 116 bp, Homo sapiens
  source 1..116: mol_type = genomic DNA
ttctgagctccggtccagccctgctgtgtgagtccctcatccccacccccgcacctgtttccaccttggctggctcaaccaacaaacttcggccccggtccgcccggtgctcaccc

SEQ ID NO: 68 - DNA, 93 bp, Homo sapiens
  source 1..93: mol_type = genomic DNA
cggtggaccgcgtgttcccgtgtggcactcacaccaggcagtgagaagatgtgtgtggctctgagctgacggtagcctgcgtgttcccgtccc

SEQ ID NO: 69 - DNA, 202 bp, Homo sapiens
  source 1..202: mol_type = genomic DNA
cccccggcggccgcgcccacgccaacccaccggcgcacatcggccatccctcgcgcttttacgcgggagcgtccgcagggccggaaggaggcccctgccccgtccaaggctgcaccagctgccccgccgcccgcccggacccagcccagcctcattgctgacgagaccccgccctgctactccctgagctcctccgccagct

SEQ ID NO: 70 - DNA, 202 bp, Homo sapiens
  source 1..202: mol_type = genomic DNA
agaatggtgaaataaatgttcttggaaattcctacccggactggctagtccttgcggaagcagcgtccgggccctcgggtaacgtttgaagagctggcagcgtctcaggctgctgcgtggcgaaggggcggaccgggggacgggggggtgggcccttaggggctggggcgggacttccctgggcactgagtcaaagcttgag

SEQ ID NO: 71 - DNA, 202 bp, Homo sapiens
  source 1..202: mol_type = genomic DNA
atgagcgcgcagagccgcgcgggcccgggaggcaggatgtgtaggcggcgagtcctcctgcatctcgctaccgagccagaccgcaggctgcgcggcgcggcgcaggcacagccaaagtgcccgagcccgcgtccccgcgtcggtacaaaaggagaaatgaggggatgaggccgaatgagtggtttctaatcgcgagaggccc